(19) United States
(12) Patent Application Publication
(10) Pub. No.: US 2025/0129174 A1
(43) Pub. Date: Apr. 24, 2025

(54) ANTI-4-1-1BBXCCR8 ANTIBODIES AND USES THEREOF

(71) Applicant: LTZ Therapeutics, Inc, Redwood City, CA (US)

(72) Inventors: Jinze Li, Danville, CA (US); Martin Treder, Reilingen (DE); Zhenfei Ning, Shenzhen (CN); Priti Singh, Sunnyvale, CA (US); Jianhui Zhou, Fremont, CA (US); Zhentao Sheng, Sunnyvale, CA (US); Jintao Feng, Shenzhen (CN)

(21) Appl. No.: 18/896,579

(22) Filed: Sep. 25, 2024

(30) Foreign Application Priority Data
Sep. 26, 2023 (WO) PCT/CN2023/121285

(51) Int. Cl. C07K 16/28; A61K 39/00; A61P 35/00

(52) U.S. Cl. CPC C07K 16/2878; A61P 35/00; C07K 16/2866; A61K 2039/505; C07K 2317/24; C07K 2317/31; C07K 2317/622

(57) ABSTRACT

This disclosure relates to an antibody or antigen-binding fragment thereof, comprising a first antigen-binding domain that specifically binds to 4-1BB and a second antigen-binding domain that specifically binds to CCR8.

### CLAIM OF PRIORITY

This application claims priority to PCT/CN2023/121285, filed on Sep. 26, 2023. The entire contents of the foregoing are incorporated herein by reference.

### SEQUENCE LISTING

This application contains a Sequence Listing that has been submitted electronically as an XML file named 55679-0003001 ST26_SL.XML.” The XML file, created on Sep. 25, 2024, is 84,831 bytes in size. The material in the XML file is hereby incorporated by reference in its entirety.

### TECHNICAL FIELD

This disclosure relates to multispecific anti-4-1BB×CCR8 antibodies (e.g., bispecific antibodies or antigen-binding fragments thereof), and antibody drug conjugates derived therefrom.

### BACKGROUND

A bispecific antibody is an artificial protein that can simultaneously bind to two different types of antigens or two different epitopes. This dual specificity opens up a wide range of applications, including redirecting T cells to tumor cells, dual targeting of different disease mediators, and delivering payloads to targeted sites. The approval of catumaxomab (anti-EpCAM and anti-CD3) and blinatumomab (anti-CD19 and anti-CD3) has become a major milestone in the development of bispecific antibodies.

As bispecific antibodies have various applications, there is a need to continue developing various therapeutics based on bispecific antibodies.

### SUMMARY

This disclosure relates to multispecific (e.g., bispecific) antibodies or antigen-binding fragments thereof, wherein the antibodies or antigen-binding fragments thereof specifically bind to 4-1BB and C—C motif chemokine receptor 8 (CCR8).

The CCR8 of the present invention includes those derived from mice, rats, hamsters, guinea pigs, dogs, pigs, and primate mammals including monkeys and humans. Human CCR8 is preferred.

The 4-1BB of the present invention includes those derived from mice, rats, hamsters, guinea pigs, dogs, pigs, and primate mammals including monkeys and humans. Human 4-1BB is preferred.

Accordingly, provided herein are antibodies or antigen-binding fragments thereof, comprising: a) a first antigen-binding domain that specifically binds to 4-1BB; and b) a second antigen-binding domain that specifically binds to C—C motif chemokine receptor 8 (CCR8).

In some embodiments, the 4-1BB is human 4-1BB, and wherein the CCR8 is human CCR8.

In some embodiments, the first antigen-binding domain comprises a first heavy chain variable region (VH1) and a first light chain variable region (VL1); and the second antigen-binding domain comprises a second heavy chain variable region (VH2) and a second light chain variable region (VL2).

In some embodiments, the second antigen-binding domain is a single-chain fragment variable (scFv) domain, wherein the VH2 and VL2 are linked by a first linker.

In some embodiments, the second antigen-binding domain is connected to the N-terminus of a light chain through a second linker.

In some embodiments, the antibody or antigen-binding fragment thereof is a bispecific antibody or antigen-binding fragment thereof.

In some embodiments, the antibody or antigen-binding fragment thereof further comprises an Fc region.

In some embodiments, the C-terminus of the VH1 of the first antigen-binding domain is connected to the Fc region, optionally through a CH1 domain.

In some embodiments, the antibody comprises a first heavy chain comprising the VH1 and a first light chain comprising the VL1; and a second heavy chain comprising the VH2 and a second light chain comprising the VL2.

In some embodiments, the Fc region is an Fc region of human IgG1, IgG2, IgG3, or lgG4.

In some embodiments, the Fc region is an Fc region of human IgG1 or IgG2.

In some embodiments, the Fc region is a wild type Fc, or an Fc with enhanced ADCC activity, or an Fc with enhanced ADCP activity.

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, 3 amino acid sequences and the selected VL1 CDRs 1, 2, 3 amino acid sequences are one of the following:
(1) the selected VH1 CDRs 1, 2, 3 amino acid sequences are set forth in SEQ ID NOs: 1, 2 and 3, respectively, and the selected VL1 CDRs 1, 2, 3 amino acid sequences are set forth in SEQ ID NOs: 4, 5 and 6, respectively;
(2) the selected VH1 CDRs 1, 2, 3 amino acid sequences are set forth in SEQ ID NOs: 24, 25 and 26, respectively, and the selected VL1 CDRs 1, 2, 3 amino acid sequences are set forth in SEQ ID NOs: 27, 28 and 29, respectively;
(3) the selected VH1 CDRs 1, 2, 3 amino acid sequences are set forth in SEQ ID NOs: 50, 51 and 52, respectively, and the selected VL1 CDRs 1, 2, 3 amino acid sequences are set forth in SEQ ID NOs: 53, 54 and 55, respectively; and
(4) the selected VH1 CDRs 1, 2, 3 amino acid sequences are set forth in SEQ ID NOs: 68, 69 and 70, respectively, and the selected VL1 CDRs 1, 2, 3 amino acid sequences are set forth in SEQ ID NOs: 71, 72 and 73, respectively.

In some embodiments, the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence, wherein the selected VH2 CDRs 1, 2, 3 amino acid sequences and the selected VL2 CDRs 1, 2, 3 amino acid sequences are one of the following:
(1) the selected VH2 CDRs 1, 2, 3 amino acid sequences are set forth in SEQ ID NOs: 11, 12 and 13, respectively, and the selected VL2 CDRs 1, 2, 3 amino acid sequences are set forth in SEQ ID NOs: 14, 15 and 16, respectively;
(2) the selected VH2 CDRs 1, 2, 3 amino acid sequences are set forth in SEQ ID NOs: 36, 37 and 38, respectively, and the selected VL2 CDRs 1, 2, 3 amino acid sequences are set forth in SEQ ID NOs: 39, 40 and 41, respectively
(3) the selected VH2 CDRs 1, 2, 3 amino acid sequences are set forth in SEQ ID NOs: 58, 59 and 60, respectively, and the selected VL2 CDRs 1, 2, 3 amino acid sequences are set forth in SEQ ID NOs: 61, 62 and 63, respectively; and
(4) the selected VH2 CDRs 1, 2, 3 amino acid sequences are set forth in SEQ ID NOs: 76, 77 and 78, respectively, and the selected VL2 CDRs 1, 2, 3 amino acid sequences are set forth in SEQ ID NOs: 79, 80 and 81, respectively.

In some embodiments, the selected VH1 CDRs 1, 2, 3 amino acid sequences are set forth in SEQ ID NOs: 1, 2 and 3, respectively, the selected VL1 CDRs 1, 2, 3 amino acid sequences are set forth in SEQ ID NOs: 4, 5 and 6, respectively; the selected VH2 CDRs 1, 2, 3 amino acid sequences are set forth in SEQ ID NOs: 11 12 and 13, respectively, and the selected VL2 CDRs 1, 2, 3 amino acid sequences are set forth in SEQ ID NOs: 14, 15 and 16, respectively.

The present disclosure is also related to antibodies or antigen-binding fragments thereof that cross-competes with any one of the antibodies or antigen-binding fragments thereof described herein.

The present disclosure is also related to methods of treating a subject having cancer, the method comprising administering a therapeutically effective amount of a composition comprising any one of the antibodies or antigen-binding fragments thereof described herein to the subject.

In some embodiments, the cancer is a solid tumor or a blood tumor.

In some embodiments, the cancer is breast cancer, uterine corpus cancer, cervical cancer, ovary cancer, prostate cancer, lung cancer, stomach cancer, non-small cell lung cancer, spleen cancer, head and neck squamous cell carcinoma, esophageal cancer, bladder cancer, melanoma, colorectal cancer, kidney cancer, non-Hodgkin lymphoma, urothelial cancer, sarcoma, blood cell cancer such as leukemia or lymphoma, bile duct carcinoma, gallbladder carcinoma, thyroid carcinoma, testicular carcinoma, thymic carcinoma, or hepatocarcinoma.

In some embodiments, the cancer is breast cancer, uterine corpus cancer, ovary cancer, lung cancer, colorectal cancer, kidney cancer and sarcoma.

In some embodiments, the cancer is colon cancer.

In some embodiments, the subject is further treated with an effective amount of an anti-OX40 antibody, an anti-PD-1 antibody, an anti-CTLA4 antibody, an anti-CD40 antibody, or an anti-PD-L1 antibody.

The present disclosure is also related to methods of decreasing the rate of tumor growth, the method comprising contacting a tumor cell with an effective amount of a composition comprising any one of the antibodies or antigen-binding fragments thereof described herein.

The present disclosure is also related to methods of killing a tumor cell, the method comprising contacting a tumor cell with an effective amount of a composition comprising any one of the antibodies or antigen-binding fragments thereof described herein.

The present disclosure is also related to methods of increasing immune response in a subject, the method comprising administering to the subject an effective amount of a composition comprising any one of the antibodies or antigen-binding fragments thereof described herein.

The present disclosure is also related to pharmaceutical compositions comprising any one of the antibodies or antigen-binding fragments thereof described herein, and a pharmaceutically acceptable carrier.

As used herein, the term “antigen-binding domain” refers to one or more protein domain(s) (e.g., formed from amino acids from a single polypeptide or formed from amino acids from two or more polypeptides (e.g., the same or different polypeptides) that is capable of specifically binding to one or more different antigen(s) (e.g., an effector antigen or tumor antigen). In some examples, an antigen-binding domain can bind to an antigen or epitope with specificity and affinity similar to that of naturally-occurring antibodies. In some embodiments, the antigen-binding domain can be an antibody or a fragment thereof. One example of an antigen-binding domain is an antigen-binding domain formed by VH-VL. In some embodiments, an antigen-binding domain can include an alternative scaffold. In some embodiments, the antigen-binding domain is a VHH. Non-limiting examples of antigen-binding domains are described herein. Additional examples of antigen-binding domains are known in the art. In some examples, an antigen-binding domain can bind to a single antigen (e.g., one of an effector antigen and a tumor antigen).

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an antigen. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)₂, and Fv fragments.

As used herein, the term “multispecific antibody” is an antibody that includes two or more different antigen-binding domains that collectively specifically bind two or more different epitopes. The two or more different epitopes may be epitopes on the same antigen (e.g., a single polypeptide present on the surface of a cell) or on different antigens (e.g., different proteins present on the surface of the same cell or present on the surface of different cells). In some aspects, a multispecific antibody binds two different epitopes (i.e., a “bispecific antibody”). In some aspects, a multispecific antibody binds three different epitopes (i.e., a “trispecific antibody”). In some aspects, a multispecific antibody binds four different epitopes (i.e., a “quadspecific antibody”). In some aspects, a multispecific antibody binds five different epitopes (i.e., a “quintspecific antibody”). Each binding specificity may be present in any suitable valency. Non-limiting examples of multispecific antibodies are described herein.

As used herein, the term “bispecific antibody” refers to an antibody that binds to two different epitopes. The epitopes can be on the same antigen or on different antigens.

As used herein, the term “anti-4-1BB×CCR8 antibody or antigen-binding fragment thereof” refers to an antibody or antigen-binding fragment that binds to both 4-1BB and CCR8.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1G are schematics illustrating exemplary 4-1BB×CCR8 bispecific antibodies described herein.

FIG. 2 shows the dosing regimen of anti-tumor Study 1 using CT 26 tumor model. Mice were treated with 4-1BB-1×CCR8-1 bispecific antibody (BsAb), CCR8-1 mAb and 4-1BB-1 mAb, with PBS as control. The first (1^{st}), second (2^{nd}), and third (3^{rd}) doses were 1 mg/kg of each mAb or 1.33 mg/kg BsAb, while the fourth (4^{th}) dose was 10 mg/kg.

FIG. 3 shows the mean tumor volume curve in anti-tumor Study 1. Each curve represents the mean tumor volume of each treatment group.

FIGS. 4A-4D show the individual tumor volume curve in anti-tumor Study 1 using CT 26 tumor implantation model. Each curve represents the tumor volume of each individual mouse.

FIG. 5 shows the dosing regimen of anti-tumor Study 2 using CT 26 tumor implantation model. Implanted mice were treated with 4-1BB-1×CCR8-1 bispecific antibody (1.33 or 4 mg/kg), CCR8-1 mAb (1 or 3 mg/kg), and 4-1BB-1 mAb (1 or 3 mg/kg), with PBS as control.

FIGS. 6A-6B show the mean tumor volume curve in anti-tumor Study 2. Each curve represents the mean tumor volume of each treatment group. FIG. 6A shows that implanted mice were treated with 4-1BB-1×CCR8-1 bispecific antibody (1.33 mg/kg), CCR8-1 mAb (1 mg/kg), and 4-1BB-1 mAb (1 mg/kg), with PBS as control. FIG. 6B shows that implanted mice were treated with 4-1BB-1×CCR8-1 bispecific antibody (4 mg/kg), CCR8-1 mAb (1 mg/kg), and 4-1BB-1 mAb (1 mg/kg), with PBS as control.

FIGS. 7A-7F show the individual tumor volume curve in anti-tumor Study 2. Each curve represents the tumor volume of each individual mouse treated with 1 mg/kg of CCR8-1 mAb (FIG. 7A), 1 mg/kg of 4-1BB-1 mAb (FIG. 7B), 1.33 mg/kg 4-1BB-1×CCR8-1 bispecific antibody (FIG. 7C), 3 mg/kg of CCR8-1 mAb (FIG. 7D), 3 mg/kg of 4-1BB-1 mAb (FIG. 7E), or 4 mg/kg 4-1BB-1×CCR8-1 bispecific antibody (FIG. 7F).

FIG. 8 shows the mice survival percentage in anti-tumor Study 2. Mice were treated with 4-1BB-1×CCR8-1 bispecific antibody (1.33 mg/kg), CCR8-1 mAb (1 mg/kg), and 4-1BB-1 mAb (1 mg/kg), with PBS as control.

FIG. 9 shows the dosing regimen of anti-tumor Study 3 using CT26 tumor implantation model. Implanted mice were treated with 4-1BB-1×CCR8-1 bispecific antibody (1.33 mg/kg), CCR8-1 mAb (1 mg/kg), and 4-1BB-1 mAb (1 mg/kg), with PBS as control.

FIG. 10 shows the mean tumor volume curve in anti-tumor Study 3. Each curve represents the mean tumor volume of each treatment group that was treated with 4-1BB-2×CCR8-1 bispecific antibody (1.33 mg/kg), CCR8-1 mAb (1 mg/kg), or 4-1BB-2 mAb (1 mg/kg), with PBS as control.

FIGS. 11A-11D show the individual tumor volume curve in anti-tumor Study 3. Each curve represents the tumor volume of each individual mouse treated with PBS (FIG. 11A), 1 mg/kg of CCR8-1 mAb (FIG. 11B), 1 mg/kg of 4-1BB-2 mAb (FIG. 11C), 1.33 mg/kg 4-1BB-1×CCR8-2 bispecific antibody (FIG. 11D).

FIGS. 12A-12B show the binding effectivity of 4-1BB×CCR8 bispecific antibodies to CHO cells expressing human 4-1BB. The mean fluorescence intensity (MFI of PE, FIG. 12A) of each antibody and the positive percent of cells binding to each antibody (FIG. 12B) were analyzed.

FIGS. 13A-13B show the binding effectivity of 4-1BB×CCR8 bispecific antibodies to CHO cells expressing human CCR8. The mean fluorescence intensity (MFI of PE, FIG. 13A) of each antibody and the positive percent of cells binding to each antibody (FIG. 13B) were analyzed.

FIG. 14 shows the binding effectivity of 4-1BB×CCR8 bispecific antibodies to CHO cells co-expressing human 4-1BB and human CCR8.

FIGS. 15A-15B show the cell killing of CHO cells co-expressing human CCR8 and 4-1BB by PBMCs, mediated by 4-1BB×CCR8 bispecific antibodies.

### DETAILED DESCRIPTION

A multispecific antibody or antigen-binding fragment thereof is an artificial protein that can simultaneously bind to two or more than two different epitopes (e.g., on two different antigens). In some embodiments, the multispecific antibody is a bispecific antibody. A bispecific antibody or antigen-binding fragment thereof can have two arms. Each arm can have one heavy chain variable region and one light chain variable region, forming an antigen-binding domain (or an antigen-binding region). The two arms can bind to two different antigens. In some embodiments, additional antigen-binding domains can be added to a monoclonal antibody (e.g., to the C-terminus or N-terminus of the light chain or the heavy chain).

This disclosure relates to multispecific (e.g., bispecific) antibodies or antigen-binding fragments thereof, comprising a first antigen-binding domain that specifically binds to 4-1BB and a second antigen-binding domain that specifically binds to C—C motif chemokine receptor 8 (CCR8).

### Anti-4-1BB×CCR8 Antibody

Human 4-1BB (also known as tumor necrosis factor receptor superfamily member 9, TNFRSF59 and CD137) is a tumor necrosis factor (TNF) receptor superfamily member (TNF superfamily member 9) expressed on number of immune cells including activated T cells, B cells, dendritic cells (DC), activated natural killer (NK) cells, as well as nonimmune cells, such as activated endothelial cells. When activated by human 4-1BB ligand, human 4-1BB co-stimulates T cells, thereby providing an effective T cell immune response and generating immune memory by inducing multiple signaling cascades.

4-1BB is a member of the TNF receptor superfamily, and is an activation-induced T-cell costimulatory molecule. Signaling via 4-1BB upregulates survival genes, enhances cell division, induces cytokine production, and prevents activation-induced cell death in T cells. The importance of the 4-1BB pathway has been underscored in a number of diseases, including cancer.

Signaling via 4-1BB by agonistic mAb, soluble 4-1BBL, or cell lines expressing 4-1BBL in the presence of anti-T-cell-receptor Ab has been shown to cause T-cell expansion, cytokine induction, and upregulation of antiapoptotic genes, and to prevent activation-induced cell death. Kinetic studies and expression analyses have revealed that although levels of 4-1BB expression are comparable in CD4+ and CD8+ T cells, CD8+ T cells have a considerably higher proliferative potential than CD4+ T cells. A similar effect was observed in vivo. In addition to its effects on T cells, ligation of the 4-1BB receptor on DCs and macrophages induces the production of IL-12 and IL-8, respectively, by these cells.

To date, the role of 4-1BB signaling has been studied in several autoimmune processes, including rheumatoid arthritis, experimental autoimmune encephalomyelitis, and systemic lupus erythematosus. There is a dual immunoregulatory effects of 4-1BB signaling.

A detailed review of 4-1BB and its functions can be found in e.g., Vinay et al., Mol Cancer Ther. 2012 May; 11 (5): 1062-70; and Bartkowiak and Curran 2015, Frontiers in Oncology, 5:117. C—C motif chemokine receptor 8 (CCR8) is a protein found mainly on the surface of a highly immunosuppressive subset of regulatory T cells (Tregs) within tumors. It is a regulator of immune response. As a chemokine receptor, CCR8 serves as a mediator of activity in concert with its chemokine CC chemokine ligand 1 (CCL1). CCR8 expression is upregulated on a highly immunosuppressive subset of Tregs in multiple cancer types. CCR8 is found to be particularly upregulated on Tregs in the tumor microenvironment (TME), with lower levels of CCR8+ Tregs found in the blood and normal tissues. Tregs act via many mechanisms within the TME, including suppression of antigen-presenting cell (APC) function, consumption of interleukin 2 (IL-2) and production of immunosuppressive cytokines and metabolites. High Treg infiltration in the TME is correlated with poor prognosis in multiple cancer types.

Targeting CCR8 has the potential to be more specific in anti-tumor activity than other current approaches to Treg depletion. Other Treg target molecules are also found on Tregs peripherally (in the blood), in normal tissue and on effector T cells. As such, a highly selective target such as CCR8 is needed that allows for the specific depletion of tumor-infiltrating Tregs without affecting Tregs in the blood and normal tissues or destroying effector T cells.

More information related to CCR8 can be found e.g., in Li H-L et al. Clinical and prognostic significance of CC chemokine receptor type 8 protein expression in gastrointestinal stromal tumors. World J Gastroenterol. 2020; 26 (31): 4656-4668; Ohue Y, Nishikawa H. Regulatory T (Treg) cells in cancer: can Treg cells be a new therapeutic target? Cancer Sci. 2019; 110 (7): 2080-2089; Cascieri M A, Springer M S. The chemokine/chemokine-receptor family: potential and progress for therapeutic intervention. Curr Opin Chem Biol. 2000; 4(4):420-427; Campbell J R et al. Fc-optimized anti-CCR8 antibody depletes regulatory T cells in human tumor models. Cancer Res. Published online Mar. 23, 2021. doi: 10.1158/0008-5472-CAN-20-3585; Van Damme H et al. Therapeutic depletion of CCR8+tumor-infiltrating regulatory T cells elicits antitumor immunity and synergizes with anti-PD-1 therapy. J Immunother Cancer. 2021; 9 (2): c001749; and Barsheshet Y et al. CCR8+FOXp3+Treg cells as master drivers of immune regulation. Proc Natl Acad Sci. 2017; 114(23):6086-6091, each of which is incorporated herein by reference in the entirety.

The disclosure provides antibodies (e.g., bispecific or multispecific) and antigen-binding fragments thereof that specifically bind to (a) 4-1BB (e.g., human 4-1BB), (b) CCR8 (e.g., human CCR8), or (c) both 4-1BB and CCR8 (e.g., human 4-1BB and human CCR8). In one aspect, the disclosure provides an anti-4-1BB×CCR8 multispecific (e.g., bispecific) antibody or antigen-binding fragment thereof, comprising: a first antigen-binding domain that specifically binds to 4-1BB; and a second antigen-binding domain that specifically binds to CCR8.

In some embodiments, the first antigen-binding domain comprises a first heavy chain variable region (VH1) and a first light chain variable region (VL1); and the second antigen-binding domain comprises a second heavy chain variable region (VH2) and a second light chain variable region (VL2).

In some embodiments, the anti-4-1BB antibody or antigen-binding fragment thereof or the anti-4-1BB×CCR8 multispecific (e.g., bispecific) antibody or antigen-binding fragment comprises an antigen-binding domain that binds to 4-1BB (e.g., human 4-1BB).

In some embodiments, the antigen-binding domain that binds to 4-1BB (e.g., human 4-1BB) comprises a first heavy chain variable region (VH1) and a first light chain variable region (VL1). In some embodiments, the anti-CCR8 antibody or antigen-binding fragment thereof or the anti-4-1BB×CCR8 multispecific (e.g., bispecific) antibody or antigen-binding fragment comprises an antigen-binding domain that binds to CCR8 (e.g., human CCR8).

In some embodiments, the antigen-binding domain that binds to CCR8 (e.g., human CCR8) comprises a second heavy chain variable region (VH2) and a second light chain variable region (VL2).

In some embodiments, the first heavy chain variable region (VH1) comprises complementarity determining regions (CDRs) 1, 2, and 3, wherein the VH1 CDR1 region comprises an amino acid sequence that is at least 80%, 90%, or 100% identical to a selected VH1 CDR1 amino acid sequence, the VH1 CDR2 region comprises an amino acid sequence that is at least 80%, 90%, or 100% identical to a selected VH1 CDR2 amino acid sequence, and the VH1 CDR3 region comprises an amino acid sequence that is at least 80%, 90%, or 100% identical to a selected VH1 CDR3 amino acid sequence; and the first light chain variable region (VL1) comprises CDRs 1, 2, and 3, wherein the VL1 CDR1 region comprises an amino acid sequence that is at least 80%, 90%, or 100% identical to a selected VL1 CDR1 amino acid sequence, the VL1 CDR2 region comprises an amino acid sequence that is at least 80%, 90%, or 100% identical to a selected VL1 CDR2 amino acid sequence, and the VL1 CDR3 region comprises an amino acid sequence that is at least 80%, 90%, or 100% identical to a selected VL1 CDR3 amino acid sequence, wherein the selected VH1 CDRs 1, 2, 3 amino acid sequences and the selected VL1 CDRs 1, 2, 3 amino acid sequences are one of the following:
(1) the selected VH1 CDRs 1, 2, 3 amino acid sequences are set forth in SEQ ID NOs: 1, 2 and 3, respectively, and the selected VL1 CDRs 1, 2, 3 amino acid sequences are set forth in SEQ ID NOs: 4, 5 and 6, respectively;
(2) the selected VH1 CDRs 1, 2, 3 amino acid sequences are set forth in SEQ ID NOs: 24, 25 and 26, respectively, and the selected VL1 CDRs 1, 2, 3 amino acid sequences are set forth in SEQ ID NOs: 27, 28 and 29, respectively;
(3) the selected VH1 CDRs 1, 2, 3 amino acid sequences are set forth in SEQ ID NOs: 50, 51 and 52, respectively, and the selected VL1 CDRs 1, 2, 3 amino acid sequences are set forth in SEQ ID NOs: 53, 54 and 55, respectively; and
(4) the selected VH1 CDRs 1, 2, 3 amino acid sequences are set forth in SEQ ID NOs: 68, 69 and 70, respectively, and the selected VL1 CDRs 1, 2, 3 amino acid sequences are set forth in SEQ ID NOs: 71, 72 and 73, respectively.

In some embodiments, the second heavy chain variable region (VH2) comprises CDRs 1, 2, and 3, wherein the VH2 CDR1 region comprises an amino acid sequence that is at least 80%, 90%, or 100% identical to a selected VH2 CDR1 amino acid sequence, the VH2 CDR2 region comprises an amino acid sequence that is at least 80%, 90%, or 100% identical to a selected VH2 CDR2 amino acid sequence, and the VH2 CDR3 region comprises an amino acid sequence that is at least 80%, 90%, or 100% identical to a selected VH2 CDR3 amino acid sequence; and the second light chain variable region (VL2) comprises CDRs 1, 2, and 3, wherein the VL2 CDR1 region comprises an amino acid sequence that is at least 80%, 90%, or 100% identical to a selected VL2 CDR1 amino acid sequence, the VL2 CDR2 region comprises an amino acid sequence that is at least 80%, 90%, or 100% identical to a selected VL2 CDR2 amino acid sequence, and the VL2 CDR3 region comprises an amino acid sequence that is at least 80%, 90%, or 100% identical to a selected VL2 CDR3 amino acid sequence, wherein the selected VH2 CDRs 1, 2, 3 amino acid sequences and the selected VL2 CDRs 1, 2, 3 amino acid sequences are one of the following:
(1) the selected VH2 CDRs 1, 2, 3 amino acid sequences are set forth in SEQ ID NOs: 11, 12 and 13, respectively, and the selected VL2 CDRs 1, 2, 3 amino acid sequences are set forth in SEQ ID NOs: 14, 15 and 16, respectively;
(2) the selected VH2 CDRs 1, 2, 3 amino acid sequences are set forth in SEQ ID NOs: 36, 37 and 38, respectively, and the selected VL2 CDRs 1, 2, 3 amino acid sequences are set forth in SEQ ID NOs: 39, 40 and 41, respectively
(3) the selected VH2 CDRs 1, 2, 3 amino acid sequences are set forth in SEQ ID NOs: 58, 59 and 60, respectively, and the selected VL2 CDRs 1, 2, 3 amino acid sequences are set forth in SEQ ID NOs: 61, 62 and 63, respectively; and
(4) the selected VH2 CDRs 1, 2, 3 amino acid sequences are set forth in SEQ ID NOs: 76, 77 and 78, respectively, and the selected VL2 CDRs 1, 2, 3 amino acid sequences are set forth in SEQ ID NOs: 79, 80 and 81, respectively.

In some embodiments, the selected VH1 CDRs 1, 2, 3 amino acid sequences are set forth in SEQ ID NOs: 1, 2 and 3, respectively, and the selected VL1 CDRs 1, 2, 3 amino acid sequences are set forth in SEQ ID NOs: 4, 5 and 6, respectively; and the selected VH2 CDRs 1, 2, 3 amino acid sequences are set forth in SEQ ID NOs: 11, 12 and 13, respectively, and the selected VL2 CDRs 1, 2, 3 amino acid sequences are set forth in SEQ ID NOs: 14, 15 and 16, respectively.

In some embodiments, the selected VH1 CDRs 1, 2, 3 amino acid sequences are set forth in SEQ ID NOs: 1, 2 and 3, respectively, and the selected VL1 CDRs 1, 2, 3 amino acid sequences are set forth in SEQ ID NOs: 4, 5 and 6, respectively; and the selected VH2 CDRs 1, 2, 3 amino acid sequences are set forth in SEQ ID NOs: 36, 37 and 38, respectively, and the selected VL2 CDRs 1, 2, 3 amino acid sequences are set forth in SEQ ID NOs: 39, 40 and 41, respectively.

In some embodiments, the selected VH1 CDRs 1, 2, 3 amino acid sequences are set forth in SEQ ID NOs: 1, 2 and 3, respectively, and the selected VL1 CDRs 1, 2, 3 amino acid sequences are set forth in SEQ ID NOs: 4, 5 and 6, respectively; and the selected VH2 CDRs 1, 2, 3 amino acid sequences are set forth in SEQ ID NOs: 58, 59 and 60, respectively, and the selected VL2 CDRs 1, 2, 3 amino acid sequences are set forth in SEQ ID NOs: 61, 62 and 63, respectively.

In some embodiments, the selected VH1 CDRs 1, 2, 3 amino acid sequences are set forth in SEQ ID NOs: 1, 2 and 3, respectively, and the selected VL1 CDRs 1, 2, 3 amino acid sequences are set forth in SEQ ID NOs: 4, 5 and 6, respectively; and the selected VH2 CDRs 1, 2, 3 amino acid sequences are set forth in SEQ ID NOs: 76, 77 and 78, respectively, and the selected VL2 CDRs 1, 2, 3 amino acid sequences are set forth in SEQ ID NOs: 79, 80 and 81, respectively.

In some embodiments, the selected VH1 CDRs 1, 2, 3 amino acid sequences are set forth in SEQ ID NOs: 24, 25 and 26, respectively, and the selected VL1 CDRs 1, 2, 3 amino acid sequences are set forth in SEQ ID NOs: 27, 28 and 29, respectively; and the selected VH2 CDRs 1, 2, 3 amino acid sequences are set forth in SEQ ID NOs: 11, 12 and 13, respectively, and the selected VL2 CDRs 1, 2, 3 amino acid sequences are set forth in SEQ ID NOs: 14, 15 and 16, respectively.

In some embodiments, the selected VH1 CDRs 1, 2, 3 amino acid sequences are set forth in SEQ ID NOs: 24, 25 and 26, respectively, and the selected VL1 CDRs 1, 2, 3 amino acid sequences are set forth in SEQ ID NOs: 27, 28 and 29, respectively; and the selected VH2 CDRs 1, 2, 3 amino acid sequences are set forth in SEQ ID NOs: 36, 37 and 38, respectively, and the selected VL2 CDRs 1, 2, 3 amino acid sequences are set forth in SEQ ID NOs: 39, 40 and 41, respectively.

In some embodiments, the selected VH1 CDRs 1, 2, 3 amino acid sequences are set forth in SEQ ID NOs: 24, 25 and 26, respectively, and the selected VL1 CDRs 1, 2, 3 amino acid sequences are set forth in SEQ ID NOs: 27, 28 and 29, respectively; and the selected VH2 CDRs 1, 2, 3 amino acid sequences are set forth in SEQ ID NOs: 58, 59 and 60, respectively, and the selected VL2 CDRs 1, 2, 3 amino acid sequences are set forth in SEQ ID NOs: 61, 62 and 63, respectively.

In some embodiments, the selected VH1 CDRs 1, 2, 3 amino acid sequences are set forth in SEQ ID NOs: 24, 25 and 26, respectively, and the selected VL1 CDRs 1, 2, 3 amino acid sequences are set forth in SEQ ID NOs: 27, 28 and 29, respectively; and the selected VH2 CDRs 1, 2, 3 amino acid sequences are set forth in SEQ ID NOs: 76, 77 and 78, respectively, and the selected VL2 CDRs 1, 2, 3 amino acid sequences are set forth in SEQ ID NOs: 79, 80 and 81, respectively.

In some embodiments, the selected VH1 CDRs 1, 2, 3 amino acid sequences are set forth in SEQ ID NOs: 50, 51 and 52, respectively, and the selected VL1 CDRs 1, 2, 3 amino acid sequences are set forth in SEQ ID NOs: 53, 54 and 55, respectively; and the selected VH2 CDRs 1, 2, 3 amino acid sequences are set forth in SEQ ID NOs: 11, 12 and 13, respectively, and the selected VL2 CDRs 1, 2, 3 amino acid sequences are set forth in SEQ ID NOs: 14, 15 and 16, respectively.

In some embodiments, the selected VH1 CDRs 1, 2, 3 amino acid sequences are set forth in SEQ ID NOs: 50, 51 and 52, respectively, and the selected VL1 CDRs 1, 2, 3 amino acid sequences are set forth in SEQ ID NOs: 53, 54 and 55, respectively; and the selected VH2 CDRs 1, 2, 3 amino acid sequences are set forth in SEQ ID NOs: 36, 37 and 38, respectively, and the selected VL2 CDRs 1, 2, 3 amino acid sequences are set forth in SEQ ID NOs: 39, 40 and 41, respectively.

In some embodiments, the selected VH1 CDRs 1, 2, 3 amino acid sequences are set forth in SEQ ID NOs: 50, 51 and 52, respectively, and the selected VL1 CDRs 1, 2, 3 amino acid sequences are set forth in SEQ ID NOs: 53, 54 and 55, respectively; and the selected VH2 CDRs 1, 2, 3 amino acid sequences are set forth in SEQ ID NOs: 58, 59 and 60, respectively, and the selected VL2 CDRs 1, 2, 3 amino acid sequences are set forth in SEQ ID NOs: 61, 62 and 63, respectively.

In some embodiments, the selected VH1 CDRs 1, 2, 3 amino acid sequences are set forth in SEQ ID NOs: 50, 51 and 52, respectively, and the selected VL1 CDRs 1, 2, 3 amino acid sequences are set forth in SEQ ID NOs: 53, 54 and 55, respectively; and the selected VH2 CDRs 1, 2, 3 amino acid sequences are set forth in SEQ ID NOs: 76, 77 and 78, respectively, and the selected VL2 CDRs 1, 2, 3 amino acid sequences are set forth in SEQ ID NOs: 79, 80 and 81, respectively.

In some embodiments, the selected VH1 CDRs 1, 2, 3 amino acid sequences are set forth in SEQ ID NOs: 68, 69, and 70, respectively, and the selected VL1 CDRs 1, 2, 3 amino acid sequences are set forth in SEQ ID NOs: 71, 72 and 73, respectively; and the selected VH2 CDRs 1, 2, 3 amino acid sequences are set forth in SEQ ID NOs: 11, 12 and 13, respectively, and the selected VL2 CDRs 1, 2, 3 amino acid sequences are set forth in SEQ ID NOs: 14, 15 and 16, respectively.

In some embodiments, the selected VH1 CDRs 1, 2, 3 amino acid sequences are set forth in SEQ ID NOs: 68, 69, and 70, respectively, and the selected VL1 CDRs 1, 2, 3 amino acid sequences are set forth in SEQ ID NOs: 71, 72 and 73, respectively and the selected VH2 CDRs 1, 2, 3 amino acid sequences are set forth in SEQ ID NOs: 36, 37 and 38, respectively, and the selected VL2 CDRs 1, 2, 3 amino acid sequences are set forth in SEQ ID NOs: 39, 40 and 41, respectively.

In some embodiments, the selected VH1 CDRs 1, 2, 3 amino acid sequences are set forth in SEQ ID NOs: 68, 69, and 70, respectively, and the selected VL1 CDRs 1, 2, 3 amino acid sequences are set forth in SEQ ID NOs: 71, 72 and 73, respectively and the selected VH2 CDRs 1, 2, 3 amino acid sequences are set forth in SEQ ID NOs: 58, 59 and 60, respectively, and the selected VL2 CDRs 1, 2, 3 amino acid sequences are set forth in SEQ ID NOs: 61, 62 and 63, respectively.

In some embodiments, the selected VH1 CDRs 1, 2, 3 amino acid sequences are set forth in SEQ ID NOs: 68, 69, and 70, respectively, and the selected VL1 CDRs 1, 2, 3 amino acid sequences are set forth in SEQ ID NOs: 71, 72 and 73, respectively; and the selected VH2 CDRs 1, 2, 3 amino acid sequences are set forth in SEQ ID NOs: 76, 77 and 78, respectively, and the selected VL2 CDRs 1, 2, 3 amino acid sequences are set forth in SEQ ID NOs: 79, 80 and 81, respectively.

In some embodiments, the VH1 comprises an amino acid sequence that is at least 80%, 85%, 90%, 95%, 99%, or 100% identical to a selected VH sequence, and the VL1 comprises an amino acid sequence that is at least 80%, 85%, 90%, 95%, 99%, or 100% identical to a selected VL sequence, wherein the selected VH sequence is SEQ ID NO: 7, 30, 56 or 74, and the selected VL sequence is SEQ ID NO: 8, 31, 57, or 75.

In some embodiments, the VH2 comprises an amino acid sequence that is at least 80%, 85%, 90%, 95%, 99%, or 100% identical to a selected VH sequence, and the VL2 comprises an amino acid sequence that is at least 80%, 85%, 90%, 95%, 99%, or 100% identical to a selected VL sequence, wherein the selected VH sequence is SEQ ID NOs: 17, 42, 64, or 82 and the selected VL sequence is SEQ ID NO: 18, 43, 65, or 83.

In some embodiments, the first heavy chain variable region comprises a sequence that is at least 80%, 85%, 90%, 95%, 99%, or 100% identical to SEQ ID NO: 7, the first light chain variable region comprises a sequence that is at least 80%, 85%, 90%, 95%, 99%, or 100% identical to SEQ ID NO: 8, the second heavy chain variable region comprises a sequence that is at least 80%, 85%, 90%, 95%, 99%, or 100% identical to SEQ ID NO: 17, and the second light chain variable region comprises a sequence that is at least 80%, 85%, 90%, 95%, 99%, or 100% identical to SEQ ID NO: 18.

In some embodiments, the first heavy chain variable region comprises a sequence that is at least 80%, 85%, 90%, 95%, 99%, or 100% identical to SEQ ID NO: 7, the first light chain variable region comprises a sequence that is at least 80%, 85%, 90%, 95%, 99%, or 100% identical to SEQ ID NO: 8, the second heavy chain variable region comprises a sequence that is at least 80%, 85%, 90%, 95%, 99%, or 100% identical to SEQ ID NO: 42, and the second light chain variable region comprises a sequence that is at least 80%, 85%, 90%, 95%, 99%, or 100% identical to SEQ ID NO: 43.

In some embodiments, the first heavy chain variable region comprises a sequence that is at least 80%, 85%, 90%, 95%, 99%, or 100% identical to SEQ ID NO: 7, the first light chain variable region comprises a sequence that is at least 80%, 85%, 90%, 95%, 99%, or 100% identical to SEQ ID NO: 8, the second heavy chain variable region comprises a sequence that is at least 80%, 85%, 90%, 95%, 99%, or 100% identical to SEQ ID NO: 64, and the second light chain variable region comprises a sequence that is at least 80%, 85%, 90%, 95%, 99%, or 100% identical to SEQ ID NO: 65.

In some embodiments, the first heavy chain variable region comprises a sequence that is at least 80%, 85%, 90%, 95%, 99%, or 100% identical to SEQ ID NO: 7, the first light chain variable region comprises a sequence that is at least 80%, 85%, 90%, 95%, 99%, or 100% identical to SEQ ID NO: 8, the second heavy chain variable region comprises a sequence that is at least 80%, 85%, 90%, 95%, 99%, or 100% identical to SEQ ID NO: 82, and the second light chain variable region comprises a sequence that is at least 80%, 85%, 90%, 95%, 99%, or 100% identical to SEQ ID NO: 83.

In some embodiments, the first heavy chain variable region comprises a sequence that is at least 80%, 85%, 90%, 95%, 99%, or 100% identical to SEQ ID NO: 30, the first light chain variable region comprises a sequence that is at least 80%, 85%, 90%, 95%, 99%, or 100% identical to SEQ ID NO: 31, the second heavy chain variable region comprises a sequence that is at least 80%, 85%, 90%, 95%, 99%, or 100% identical to SEQ ID NO: 17, and the second light chain variable region comprises a sequence that is at least 80%, 85%, 90%, 95%, 99%, or 100% identical to SEQ ID NO: 18.

In some embodiments, the first heavy chain variable region comprises a sequence that is at least 80%, 85%, 90%, 95%, 99%, or 100% identical to SEQ ID NO: 30, the first light chain variable region comprises a sequence that is at least 80%, 85%, 90%, 95%, 99%, or 100% identical to SEQ ID NO: 31, the second heavy chain variable region comprises a sequence that is at least 80%, 85%, 90%, 95%, 99%, or 100% identical to SEQ ID NO: 42, and the second light chain variable region comprises a sequence that is at least 80%, 85%, 90%, 95%, 99%, or 100% identical to SEQ ID NO: 43.

In some embodiments, the first heavy chain variable region comprises a sequence that is at least 80%, 85%, 90%, 95%, 99%, or 100% identical to SEQ ID NO: 30, the first light chain variable region comprises a sequence that is at least 80%, 85%, 90%, 95%, 99%, or 100% identical to SEQ ID NO: 31, the second heavy chain variable region comprises a sequence that is at least 80%, 85%, 90%, 95%, 99%, or 100% identical to SEQ ID NO: 64, and the second light chain variable region comprises a sequence that is at least 80%, 85%, 90%, 95%, 99%, or 100% identical to SEQ ID NO: 65.

In some embodiments, the first heavy chain variable region comprises a sequence that is at least 80%, 85%, 90%, 95%, 99%, or 100% identical to SEQ ID NO: 30, the first light chain variable region comprises a sequence that is at least 80%, 85%, 90%, 95%, 99%, or 100% identical to SEQ ID NO: 31, the second heavy chain variable region comprises a sequence that is at least 80%, 85%, 90%, 95%, 99%, or 100% identical to SEQ ID NO: 82, and the second light chain variable region comprises a sequence that is at least 80%, 85%, 90%, 95%, 99%, or 100% identical to SEQ ID NO: 83.

In some embodiments, the first heavy chain variable region comprises a sequence that is at least 80%, 85%, 90%, 95%, 99%, or 100% identical to SEQ ID NO: 56, the first light chain variable region comprises a sequence that is at least 80%, 85%, 90%, 95%, 99%, or 100% identical to SEQ ID NO: 57, the second heavy chain variable region comprises a sequence that is at least 80%, 85%, 90%, 95%, 99%, or 100% identical to SEQ ID NO: 17, and the second light chain variable region comprises a sequence that is at least 80%, 85%, 90%, 95%, 99%, or 100% identical to SEQ ID NO: 18.

In some embodiments, the first heavy chain variable region comprises a sequence that is at least 80%, 85%, 90%, 95%, 99%, or 100% identical to SEQ ID NO: 56, the first light chain variable region comprises a sequence that is at least 80%, 85%, 90%, 95%, 99%, or 100% identical to SEQ ID NO: 57, the second heavy chain variable region comprises a sequence that is at least 80%, 85%, 90%, 95%, 99%, or 100% identical to SEQ ID NO: 42, and the second light chain variable region comprises a sequence that is at least 80%, 85%, 90%, 95%, 99%, or 100% identical to SEQ ID NO: 43.

In some embodiments, the first heavy chain variable region comprises a sequence that is at least 80%, 85%, 90%, 95%, 99%, or 100% identical to SEQ ID NO: 56, the first light chain variable region comprises a sequence that is at least 80%, 85%, 90%, 95%, 99%, or 100% identical to SEQ ID NO: 57, the second heavy chain variable region comprises a sequence that is at least 80%, 85%, 90%, 95%, 99%, or 100% identical to SEQ ID NO: 64, and the second light chain variable region comprises a sequence that is at least 80%, 85%, 90%, 95%, 99%, or 100% identical to SEQ ID NO: 65.

In some embodiments, the first heavy chain variable region comprises a sequence that is at least 80%, 85%, 90%, 95%, 99%, or 100% identical to SEQ ID NO: 56, the first light chain variable region comprises a sequence that is at least 80%, 85%, 90%, 95%, 99%, or 100% identical to SEQ ID NO: 57, the second heavy chain variable region comprises a sequence that is at least 80%, 85%, 90%, 95%, 99%, or 100% identical to SEQ ID NO: 82, and the second light chain variable region comprises a sequence that is at least 80%, 85%, 90%, 95%, 99%, or 100% identical to SEQ ID NO: 83.

In some embodiments, the first heavy chain variable region comprises a sequence that is at least 80%, 85%, 90%, 95%, 99%, or 100% identical to SEQ ID NO: 74, the first light chain variable region comprises a sequence that is at least 80%, 85%, 90%, 95%, 99%, or 100% identical to SEQ ID NO: 75, the second heavy chain variable region comprises a sequence that is at least 80%, 85%, 90%, 95%, 99%, or 100% identical to SEQ ID NO: 17, and the second light chain variable region comprises a sequence that is at least 80%, 85%, 90%, 95%, 99%, or 100% identical to SEQ ID NO: 18.

In some embodiments, the first heavy chain variable region comprises a sequence that is at least 80%, 85%, 90%, 95%, 99%, or 100% identical to SEQ ID NO: 74, the first light chain variable region comprises a sequence that is at least 80%, 85%, 90%, 95%, 99%, or 100% identical to SEQ ID NO: 75, the second heavy chain variable region comprises a sequence that is at least 80%, 85%, 90%, 95%, 99%, or 100% identical to SEQ ID NO: 42, and the second light chain variable region comprises a sequence that is at least 80%, 85%, 90%, 95%, 99%, or 100% identical to SEQ ID NO: 43.

In some embodiments, the first heavy chain variable region comprises a sequence that is at least 80%, 85%, 90%, 95%, 99%, or 100% identical to SEQ ID NO: 74, the first light chain variable region comprises a sequence that is at least 80%, 85%, 90%, 95%, 99%, or 100% identical to SEQ ID NO: 75, the second heavy chain variable region comprises a sequence that is at least 80%, 85%, 90%, 95%, 99%, or 100% identical to SEQ ID NO: 64, and the second light chain variable region comprises a sequence that is at least 80%, 85%, 90%, 95%, 99%, or 100% identical to SEQ ID NO: 65.

In some embodiments, the first heavy chain variable region comprises a sequence that is at least 80%, 85%, 90%, 95%, 99%, or 100% identical to SEQ ID NO: 74, the first light chain variable region comprises a sequence that is at least 80%, 85%, 90%, 95%, 99%, or 100% identical to SEQ ID NO: 75, the second heavy chain variable region comprises a sequence that is at least 80%, 85%, 90%, 95%, 99%, or 100% identical to SEQ ID NO: 82, and the second light chain variable region comprises a sequence that is at least 80%, 85%, 90%, 95%, 99%, or 100% identical to SEQ ID NO: 83.

In some embodiments, the VH1 comprises VH1 CDR1, VH1 CDR2, and VH1 CDR3 that are identical to VH CDR1, VH CDR2, and VH CDR3 of a selected VH sequence; and the VL1 comprising VL1 CDR1, VL1 CDR2, and VL1 CDR3 that are identical to VL CDR1, VL CDR2, and VL CDR3 of a selected VL sequence, wherein the selected VH sequence is SEQ ID NO: 7, 30, 56 or 74, and the selected VL sequence is SEQ ID NO: 8, 31, 57, or 75.

In some embodiments, the VH2 comprises VH2 CDR1, VH2CDR2, and VH2 CDR3 that are identical to VH CDR1, VH CDR2, and VH CDR3 of a selected VH sequence; and the VL2 comprising VL2 CDR1, VL2 CDR2, and VL2 CDR3 that are identical to VL CDR1, VL CDR2, and VL CDR3 of a selected VL sequence, wherein the selected VH sequence is SEQ ID NO: 17, 42, 64, or 82, and the selected VL sequence is SEQ ID NO: 18, 43, 65, or 83.

In some embodiments, the CDR definition is based on Kabat, Chothia, IMGT, contact or AbM definition.

In some embodiments, the first antigen-binding domain specifically binds to human or mouse 4-1BB; and/or the second antigen-binding domain specifically binds to human or mouse CCR8.

In some embodiments, the first antigen-binding domain is a human or humanized antigen-binding domain; and/or the second antigen-binding domain is a human or humanized antigen-binding domain.

In some embodiments, the first antigen-binding domain is a single-chain variable fragment (scFV); and/or the second antigen-binding domain is a scFv.

In some embodiments, the antibody or antigen-binding fragment thereof comprises a fragment crystallizable region (Fc region). In some embodiments, the C-terminus of the VH1 of the first antigen-binding domain is connected to the Fc region. In some embodiments, the C-terminus of the VH1 of the first antigen-binding domain is connected to the Fc region, optionally through a CH1 domain.

The first antigen-binding fragment and the second antigen-binding fragment of the bi-specific antibodies or antigen-binding fragments described herein can be in any suitable structure. In some embodiments, the second antigen-binding domain is a single-chain fragment variable (scFv) domain comprising a light chain variable domain (VL) and a heavy chain variable domain (VH) linked by a first linker.

In some embodiments, the second antigen-binding domain is linked to the N-terminus of a light chain. In some embodiments, the second antigen-binding domain is linked to the N-terminus of the first light chain variable domain (VL1). In some embodiments, the C-terminus of the second antigen-binding domain (e.g., a scFv) is linked to the N-terminus of the first light chain variable domain (VL1). In some embodiments, the second antigen-binding domain (e.g., a scFv) is linked to the N-terminus of a light chain, optionally through a second linker. In some embodiments, the second antigen-binding domain is linked to the N-terminus of a Fc, optionally through a second linker. In some embodiments, the second antigen-binding domain (e.g., a scFv) is linked to the C-terminus of a light chain, optionally through a second linker. In some embodiments, the second antigen-binding domain is linked to the C-terminus of a Fc, optionally through a second linker.

In some embodiments, the anti-4-1BB×CCR8 antibody comprises 1, 2, 3, 4, 5, 6, 7, 8, 9, 10, or more than 10 anti-4-1BB antigen binding domains and 1, 2, 3, 4, 5, 6, 7, 8, 9, 10, or more than 10 anti-4-CCR8 antigen binding domains.

In some embodiments, an anti-CCR8 antigen binding domain (e.g., scFv) is linked to the N-terminus of the heavy chain or the light chain. In some embodiments, an anti-CCR8 antigen binding domain is linked to the C-terminus of the heavy chain or the light chain.

In some embodiments, an anti-4-1BB antigen binding domain (e.g., scFv) is linked to the N-terminus of the heavy chain or the light chain. In some embodiments, an anti-4-1BB antigen binding domain is linked to the C-terminus of the heavy chain or the light chain.

In some embodiments, the anti-4-1BB×CCR8 antibody includes (1) a first polypeptide, including, optionally from N terminus to C terminus, a scFv, a VL, and a CL; (2) a second polypeptide including, optionally from N terminus to C terminus, a VH, and a CH1; (3) a third polypeptide including, optionally from N terminus to C terminus, a VH, and a CH1, wherein the third polypeptide further optionally associates with the second polypeptide through one or more disulfide bonds; (4) a fourth polypeptide including, optionally from N terminus to C terminus, a scFv, a VL, and a CL. In some embodiments, the second polypeptide further includes a CH2 and a CH3. In some embodiments, the third polypeptide further includes a CH2 and a CH3.

In some embodiments, the anti-4-1BB×CCR8 antibody includes (1) a first polypeptide, including, optionally from N terminus to C terminus, a VL, a CL, and a scFv; (2) a second polypeptide including, optionally from N terminus to C terminus, a VH, and a CH1; (3) a third polypeptide including, optionally from N terminus to C terminus, a VH, and a CH1, wherein the third polypeptide further optionally associates with the second polypeptide through one or more disulfide bonds; (4) a fourth polypeptide including, optionally from N terminus to C terminus, a VL, a CL, and a scFv. In some embodiments, the second polypeptide further includes a CH2 and a CH3. In some embodiments, the third polypeptide further includes a CH2 and a CH3.

In some embodiments, the anti-4-1BB×CCR8 antibody includes (1) a first polypeptide, including, optionally from N terminus to C terminus, a VL, and a CL; (2) a second polypeptide including, optionally from N terminus to C terminus, a scFv, a VH, and a CH1; (3) a third polypeptide including, optionally from N terminus to C terminus, a scFv, a VH, and a CH1, wherein the third polypeptide further optionally associates with the second polypeptide through one or more disulfide bonds; (4) a fourth polypeptide including, optionally from N terminus to C terminus, a VL, and a CL. In some embodiments, the second polypeptide further includes a CH2 and a CH3. In some embodiments, the third polypeptide further includes a CH2 and a CH3.

In some embodiments, the anti-4-1BB×CCR8 antibody includes (1) a first polypeptide, including, optionally from N terminus to C terminus, a VL, and a CL; (2) a second polypeptide including, optionally from N terminus to C terminus, a VH, a CH1, and a scFv; (3) a third polypeptide including, optionally from N terminus to C terminus, a VH, and a CH1, and a scFv, wherein the third polypeptide further optionally associates with the second polypeptide through one or more disulfide bonds; (4) a fourth polypeptide including, optionally from N terminus to C terminus, a VL, and a CL. In some embodiments, the second polypeptide further includes a CH2 and a CH3. In some embodiments, the third polypeptide further includes a CH2 and a CH3.

In some embodiments, the VH and VL associate together and bind to CCR8, and the scFv binds to 4-1BB.

In some embodiments, the VH and VL associate together and bind to 4-1BB, and the scFv binds to CCR8.

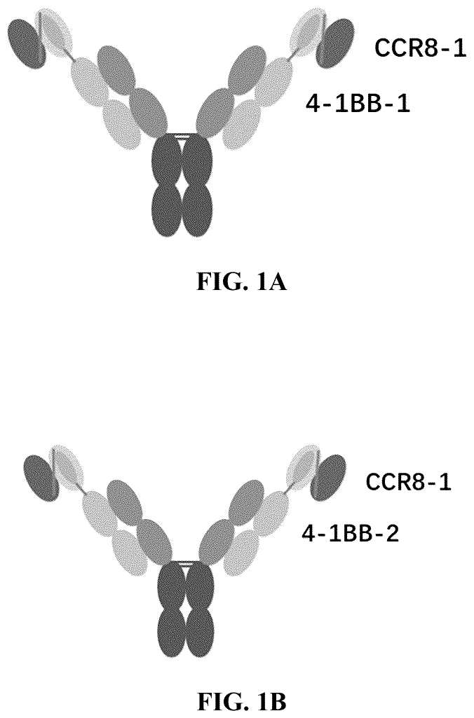

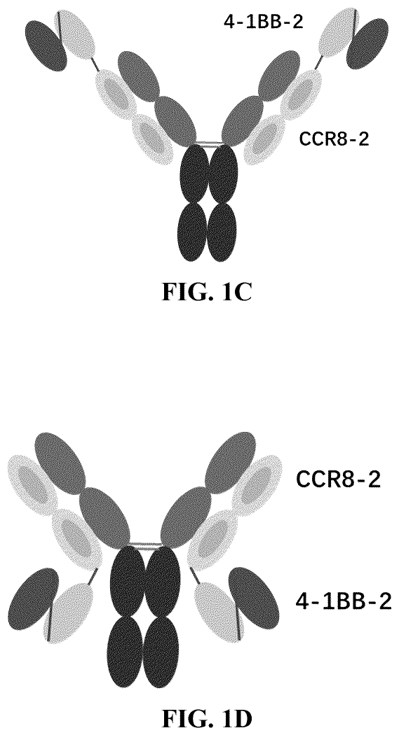

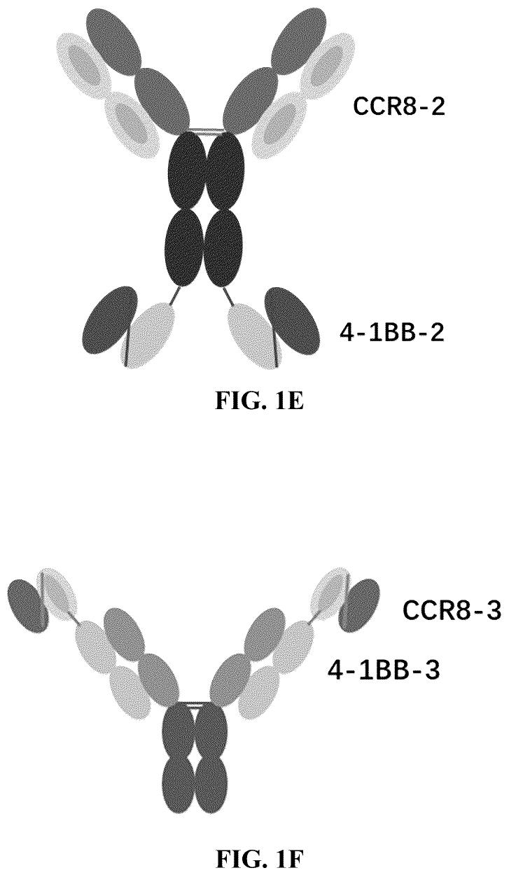

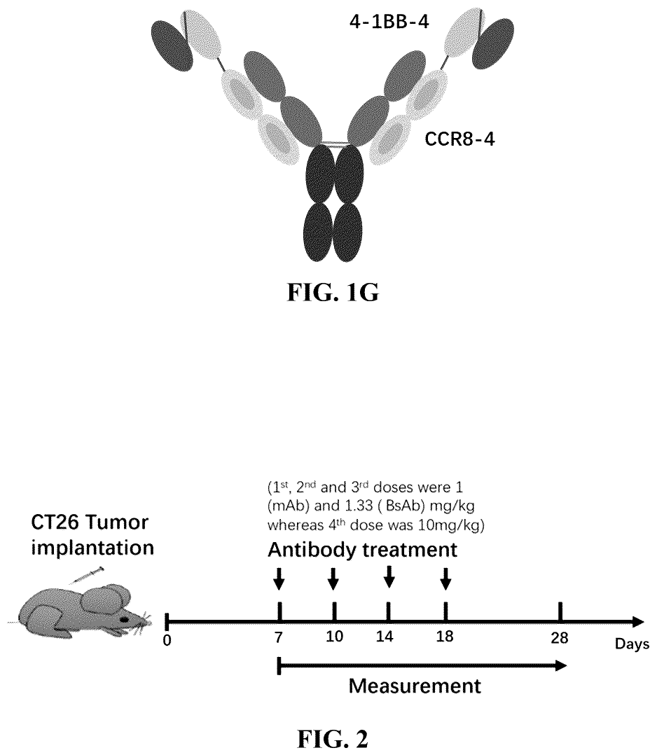

Schematic illustrations of various configuration are shown in FIGS. 1A-1G.

In some embodiments, the bispecific antibody (e.g., anti-4-1BB×CCR8 antibody) includes a first heavy chain sequence comprising the VH1, wherein the first heavy chain comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 9, 32. In some embodiments, the bispecific antibody (e.g., anti-4-1BB×CCR8 antibody) includes a first light chain sequence comprising the VL1, wherein the first light chain comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 10, 33.

In some embodiments, the bispecific antibody (e.g., anti-4-1BB×CCR8 antibody) includes a second heavy chain sequence comprising the VH2, wherein the second heavy chain comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 19. In some embodiments, the bispecific antibody (e.g., anti-4-1BB×CCR8 antibody) includes a second light chain sequence comprising the VL2, wherein the second light chain comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 20.

In some embodiments, the bispecific antibody (e.g., anti-4-1BB×CCR8 antibody) includes a heavy chain sequence comprising an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 21, 34, 44, 46, 48, 66, or 84. In some embodiments, the bispecific antibody (e.g., anti-4-1BB×CCR8 antibody) includes a light chain sequence comprising an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 22, 35, 45, 47, 49, 67, or 85. The linkers described herein can be any suitable linkers known in the art. In some embodiments, the linker includes an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 23 or 86.

The disclosure provides anti-4-1BB×CCR8 antibodies and the modified antibodies thereof, including, e.g., chimeric antibodies, humanized antibodies, and human antibodies.

The anti-4-1BB×CCR8 antibodies or antigen-binding fragments thereof can also have various forms. Many different formats of bispecific antibodies or antigen-binding fragments thereof are known in the art, and are described e.g., in Suurs, et al. “A review of bispecific antibodies and antibody constructs in oncology and clinical challenges,” Pharmacology & therapeutics (2019), which is incorporated herein by reference in the entirety.

In some embodiments, the anti-4-1BB×CCR8 antibody is a BiTe, a (scFv) 2, a nanobody, a nanobody-HSA, a DART, a TandAb, a scDiabody, a scDiabody-CH3, scFv-CH-CL-scFv, a HSAbody, scDiabody-HAS, or a tandem-scFv. In some embodiments, the anti-4-1BB×CCR8 antibody is a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv2, a scFv-KIH, a Fab-scFv-Fc, a tetravalent HCAb, a scDiabody-Fc, a Diabody-Fc, a tandem scFv-Fc, an Intrabody, a dock and lock, a lmmTAC, an IgG-IgG conjugate, a Cov-X-Body, or a scFv1-PEG-scFv2.

In some embodiments, the anti-4-1BB×CCR8 antibody can be a TrioMab. In a TrioMab, the two heavy chains are from different species, wherein different sequences restrict the heavy-light chain pairing.

In some embodiments, the anti-4-1BB×CCR8 antibody has two different heavy chains and two different light chains. Heterodimerization of heavy chains can be based on the knobs-into-holes or some other heavy chain pairing technique.

In some embodiments, CrossMAb technique can be used produce bispecific anti-4-1BB×CCR8 antibodies. CrossMAb technique can be used enforce correct light chain association in bispecific heterodimeric IgG antibodies, this technique allows the generation of various bispecific antibody formats, including bi-(1+1), tri-(2+1) and tetra-(2+2) valent bispecific antibodies, as well as non-Fc tandem antigen-binding fragment (Fab)-based antibodies. These formats can be derived from any existing antibody pair using domain crossover, without the need for the identification of common light chains, post-translational processing/in vitro chemical assembly or the introduction of a set of mutations enforcing correct light chain association. The method is described in Klein et al., “The use of CrossMAb technology for the generation of bi- and multispecific antibodies.” MAbs. Vol. 8. No. 6. Taylor & Francis, 2016, which is incorporated by reference in its entirety. In some embodiments, the CH1 in the heavy chain and the CL domain in the light chain are swapped.

The anti-4-1BB×CCR8 antibody can be a Duobody. The Fab-exchange mechanism naturally occurring in IgG4 antibodies is mimicked in a controlled matter in IgG1 antibodies, a mechanism called controlled Fab exchange. This format can ensure specific pairing between the heavy-light chains.

In Dual-variable-domain antibody (DVD-Ig), additional VH and variable light chain (VL) domain are added to each N-terminus for bispecific targeting. This format resembles the IgG-scFv, but the added binding domains are bound individually to their respective N-termini instead of a scFv to each heavy chain N-terminus.

In scFv-IgG, the two scFv are connected to the C-terminus of the heavy chain (CH3). The scFv-IgG format has two different bivalent binding sites and is consequently also called tetravalent. There are no heavy-chain and light-chain pairing problem in the scFv-IgG.

In some embodiments, the anti-4-1BB×CCR8 antibody can have a IgG-IgG format. Two intact IgG antibodies are conjugated by chemically linking the C-terminals of the heavy chains.

The anti-4-1BB×CCR8 antibody can also have a Fab-scFv-Fc format. In Fab-scFv-Fc format, a light chain, heavy chain and a third chain containing the Fc region and the scFv are assembled. It can ensure efficient manufacturing and purification.

In some embodiments, the anti-4-1BB×CCR8 antibody can be a TF. Three Fab fragments are linked by disulfide bridges. The TF format does not have an Fc region.

ADAPTIR has two scFvs bound to each side of an Fc region. It abandons the intact IgG as a basis for its construct, but conserves the Fc region to extend the half-life and facilitate purification.

Dual affinity retargeting (DART) has two peptide chains connecting the opposite fragments, thus VLA with VHB and VLB with VHA, and a sulfur bond at their C-termini fusing them together. In DART, the sulfur bond can improve stability over BiTEs.

In DART-Fc, an Fc region is attached to the DART structure. It can be generated by assembling three chains, two via a disulfide bond, as with the DART. One chain contains half of the Fc region which will dimerize with the third chain, only expressing the Fc region. The addition of Fc region enhances half-life leading to longer effective concentrations, avoiding continuous IV.

In tetravalent DART, four peptide chains are assembled. Basically, two DART molecules are created with half an Fc region and will dimerize. This format has bivalent binding to both targets, thus it is a tetravalent molecule.

Tandem diabody (TandAb) comprises two diabodies. Each diabody consists of an VHA and VLB fragment and a VHA and VLB fragment that are covalently associated. The two diabodies are linked with a peptide chain. It can improve stability over the diabody consisting of two scFvs. It has two bivalent binding sites.

The ScFv-scFv-toxin includes toxin and two scFv with a stabilizing linker. It can be used for specific delivery of payload.

In some embodiments, the anti-4-1BB×CCR8 antibody is a bispecific antibody. In some embodiments, the bispecific antibody in present disclosure is designed to be 1+1 (monovalent for each target) and has an IgG1 subtype structure. This can reduce the avidity to cells with low expression levels of 4-1BB and CCR8, and increase the avidity to cells that co-express 4-1BB and CCR8, to achieve enhanced targeting function.

In some embodiments, the anti-4-1BB×CCR8 antibodies include KIH mutations. In some embodiments, the anti-4-1BB×CCR8 antibody includes a first antigen-binding domain that specifically binds to 4-1BB, and a second antigen-binding domain that specifically binds to CCR8. In some embodiments, the first antigen-binding domain includes a heavy chain that including one or more knob mutations (a knob heavy chain), and the second antigen-binding domain includes a heavy chain including one or more hole mutations (a hole heavy chain). In some embodiments, the first antigen-binding domain includes a heavy chain that includes one or more hole mutations (a hole heavy chain), and the second antigen-binding domain that includes a heavy chain including one or more knob mutations (a knob heavy chain).

In some embodiments, the anti-4-1BB×CCR8 antibodies, or antibody fragments thereof include the combinations of anti-4-1BB and anti-CCR8 antigen-binding domains shown in Table 1.

The disclosure also provides nucleic acid comprising a polynucleotide encoding an anti-4-1BB×CCR8 antibody. The immunoglobulin heavy chain or immunoglobulin light chain in the anti-4-1BB×CCR8 antibody comprises CDRs as shown in Table 1. When the polypeptides are paired with corresponding polypeptide (e.g., a corresponding heavy chain variable region or a corresponding light chain variable region), the paired polypeptides bind to CCR8 and/or 4-1BB.

The anti-4-1BB×CCR8 antibodies can also be anti-4-1BB×CCR8 antibody variants (including derivatives and conjugates) of anti-4-1BB×CCR8 antibodies or antibody fragments. Additional anti-4-1BB×CCR8 antibodies provided herein are polyclonal, monoclonal, multispecific (multimeric, e.g., bispecific), human antibodies, chimeric antibodies (e.g., human-mouse chimera), single-chain antibodies, intracellularly-made antibodies (i.e., intrabodies), and antigen-binding fragments thereof. The anti-4-1BB×CCR8 antibodies can be of any type (e.g., IgG, IgE, IgM, IgD, IgA, and IgY), class (e.g., IgG1, IgG2, lgG3, IgG4, IgA1, and IgA2), or subclass. In some embodiments, the anti-4-1BB×CCR8 antibody or antigen-binding fragment is an IgG (e.g., IgG1 Fc region) antibody or antigen-binding fragment thereof.

Fragments of anti-4-1BB×CCR8 antibodies are suitable for use in the methods provided so long as they retain the desired affinity and specificity to both CCR8 and 4-1BB. Thus, a fragment of an anti-4-1BB×CCR8 antibody will retain an ability to bind to CCR8 and 4-1BB.

In some embodiments, the antibodies or antigen-binding fragments thereof have identical light chain variable regions.

In some embodiments, the multispecific (e.g., bispecific) anti-4-1BB×CCR8 antibody described herein kill tumor cells and/or reduces tumor size (e.g., through the activation of the 4-1 BB-related signaling pathway and the recruiting and depletion of CCR8-expressing immune cells (e.g., CCR8-expressing tumor infiltrating Tregs)) in a subject receiving the antibody. In some embodiments, about 5%, 10%, 15%, 20%, 25%, 30%, 35%, 40%, 45%, 50%, 55%, 60%, 65%, 70%, 75%, 80%, 85%, 90%, 95% or more of the tumor cells are killed by the administration of the multispecific (e.g., bispecific) anti-4-1BB×CCR8 antibody described herein.

In some embodiments, the tumor size in a subject receiving the multispecific (e.g., bispecific) anti-4-1BB×CCR8 antibody described herein is reduced by about 5%, 10%, 15%, 20%, 25%, 30%, 35%, 40%, 45%, 50%, 55%, 60%, 65%, 70%, 75%, 80%, 85%, 90%, 95% or more, compared the tumor size without the administration of the multispecific (e.g., bispecific) anti-4-1BB×CCR8 antibody. In some embodiments, the tumor size in a subject receiving the multispecific (e.g., bispecific) anti-4-1BB×CCR8 antibody described herein is reduced about 5%, 10%, 15%, 20%, 25%, 30%, 35%, 40%, 45%, 50%, 55%, 60%, 65%, 70%, 75%, 80%, 85%, 90%, 95% or more than the tumor size reduced by the administration of a monospecific antibody (e.g., a monospecific anti-4-1BB antibody or monospecific anti-CCR8 antibody).

In some embodiments, the administration of the multispecific (e.g., bispecific) anti-4-1BB×CCR8 antibody described herein increases the survival rate of the subject receiving the antibody. In some embodiments, the survival rate is increased by about 5%, 10%, 15%, 20%, 25%, 30%, 35%, 40%, 45%, 50%, 55%, 60%, 65%, 70%, 75%, 80%, 85%, 90%, 95% or more, compared the survival rate of a subject without the administration of the multispecific (e.g., bispecific) anti-4-1BB×CCR8 antibody.

In some embodiments, the antitumor effect of the anti-4-1BB×CCR8 antibody is through the modulation of Treg/Teff cells (e.g., in mice or in humans) to enhance tumor immunity and increase the survival rate.

### Antibodies and Antigen Binding Fragments Thereof

In some embodiments, the bispecific antibody (e.g., anti-4-1BB×CCR8 antibody) includes an antigen-binding domain that is derived from an anti-4-1BB antibody, and an antigen-binding domain that is derived from an anti-CCR8 antibody. These anti-4-1BB×CCR8 antibodies and antigen-binding fragments thereof can have various forms.

In general, antibodies (also called immunoglobulins) can be made up of two classes of polypeptide chains, light chains and heavy chains. A non-limiting anti-4-1BB×CCR8 antibody of the present disclosure can be an intact, four immunoglobulin chain antibody comprising two heavy chains and two light chains. The heavy chain of the anti-4-1BB×CCR8 antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain.

The hypervariable regions, known as the complementary determining regions (CDRs), form loops that comprise the principal antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding domain.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132:211-250; Martin et al., Methods Enzymol. 203:121-53 (1991); Morea et al., Biophys Chem. 68 (1-3): 9-16 (October 1997); Morea et al., J Mol Biol. 275 (2): 269-94 (January 1998); Chothia et al., Nature 342 (6252): 877-83 (December 1989); Ponomarenko and Bourne, BMC Structural Biology 7:64 (2007); each of which is incorporated herein by reference in its entirety.

The CDRs are important for recognizing an epitope of an antigen. As used herein, an “epitope” is the smallest portion of a target molecule capable of being specifically bound by the antigen-binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the anti-4-1BB×CCR8 antibody is an intact immunoglobulin molecule (e.g., IgG1, IgG2a, IgG2b, IgG3, IgM, IgD, IgE, IgA). The IgG subclasses (IgG1, IgG2, IgG3, and lgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The anti-4-1BB×CCR8 antibody can also be an immunoglobulin molecule that is derived from any species (e.g., human, rodent, mouse, rat, or camelid). The antigen-binding domain or antigen binding fragment is a portion of an antibody that retains specific binding activity of the intact antibody, i.e., any portion of an antibody that is capable of specific binding to an epitope on the intact antibody's target molecule. It includes, e.g., Fab, Fab′, F(ab′)₂, and variants of these fragments. Thus, in some embodiments, an anti-4-1BB×CCR8 antibody or antigen binding fragment thereof can comprise e.g., a scFv, a Fv, a Fd, a dAb, a bispecific antibody, a bispecific scFv, a diabody, a linear antibody, a single-chain antibody molecule, a multispecific antibody formed from antibody fragments, and any polypeptide that includes a binding domain which is, or is homologous to, an antibody binding domain. Non-limiting examples of antigen-binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the scFv in an anti-4-1BB×CCR8 antibody has two heavy chain variable domains, and two light chain variable domains. In some embodiments, the anti-4-1BB×CCR8 scFv has two antigen binding regions (Antigen binding regions: A and B), and the two antigen binding regions can bind to the respective target antigens with different affinities.

In some embodiments, the anti-4-1BB×CCR8 antibodies or antigen-binding fragments thereof can comprises one, two, or three heavy chain variable region CDRs selected from Table 1.

In some embodiments, the anti-4-1BB×CCR8 antibodies described herein can be conjugated to a therapeutic agent. The anti-4-1BB×CCR8 antibody-drug conjugate comprising the antibody or antigen-binding fragment thereof can covalently or non-covalently bind to a therapeutic agent. In some embodiments, the therapeutic agent is a cytotoxic or cytostatic agent (e.g., monomethyl auristatin E, monomethyl auristatin F, cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs). In some embodiments, the therapeutic agent is MMAE or MMAF. In some embodiments, the therapeutic agent is conjugated via a linker, e.g., a VC linker. Details of the linkers used for ADCs can be found, e.g., in Su, Z. et al. “Antibody-drug conjugates: Recent advances in linker chemistry.” Acta Pharmaceutica Sinica B (2021), which is incorporated herein by reference in its entirety.

In some embodiments, the anti-4-1BB×CCR8 antibody is a bispecific antibody. Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Any of the anti-4-1BB×CCR8 antibodies or antigen-binding fragments thereof described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an anti-4-1BB×CCR8 antibody or an antigen-binding fragment in vitro (e.g., in tissue culture or when stored as a pharmaceutical composition) or in vivo (e.g., in a human).

### Antibody Characteristics

The anti-4-1BB×CCR8 antibodies can include an anti-4-1BB antigen-binding domain and any anti-CCR8 antigen-binding domain as described herein. In some embodiments, the anti-4-1BB×CCR8 antibodies, or antigen-binding fragments thereof can initiate target cell (e.g., tumor cell, such as CT26 cell) killing, CDC, ADCC, or ADCP.

The disclosure provides anti-4-1BB×CCR8 antibodies and antigen-binding fragments thereof that can specifically bind to 4-1BB. In some embodiments, by binding to 4-1BB or activating the 4-1BB interaction with its ligands, the anti-4-1BB×CCR8 antibody can activate 4-1BB-associated signaling pathways, thus treating cancer. In some embodiments, the antibody can reduce tumor volume in a subject. Thus, in some embodiments, the anti-4-1BB×CCR8 antibody described herein is a 4-1BB agonist. In some embodiments, the anti-4-1BB×CCR8 antibody described herein is a 4-1BB antagonist.

General techniques can be used to measure the affinity of an antibody for an antigen include, e.g., ELISA, RIA, and surface plasmon resonance (SPR). Affinities can be deduced from the quotient of the kinetic rate constants (KD=koff/kon). In some implementations, the anti-4-1BB×CCR8 antibodies or antigen-binding fragments thereof can bind to 4-1BB (e.g., human 4-1BB, monkey 4-1BB, mouse 4-1BB, and/or chimeric 4-1BB) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

In some embodiments, the anti-4-1BB×CCR8 antibodies or antigen-binding fragments thereof can bind to 4-1BB (e.g., human 4-1BB, monkey 4-1BB, mouse 4-1BB, and/or chimeric 4-1BB) with a KD of less than 1×10⁻⁶ M, less than 1×10⁻⁷ M, less than 1×10⁻⁸ M, less than 1×10⁻⁹ M, or less than 1×10⁻¹⁰ M. In some embodiments, the KD is less than 50 nM, 40 nM, 30 nM, 20 nM, 10 nM, 9 nM, 8 nM, 7 nM, 6 nM, 5 nM, 4 nM, 3 nM, 2 nM, or 1 nM. In some embodiments, KD is greater than 1×10⁻⁷M, greater than 1×10⁻⁸ M, greater than 1×10⁻⁹ M, or greater than 1×10⁻¹⁰ M.

The anti-4-1BB×CCR8 antibodies or antigen-binding fragments thereof can also include an antigen-binding domain that can specifically bind to CCR8. In some embodiments, the antibodies or antigen-binding fragments thereof described herein recruit CCR8-expressing cells (e.g., CCR8-expressing Tregs) and mediate the depletion of such cells. In some embodiments, the antibody can reduce tumor volume in an animal. In some embodiments, the antibody can reduce or slow down the progression of an autoimmune disease. Thus, in some embodiments, the anti-4-1BB×CCR8 antibody described herein is a CCR8 agonist. In some embodiments, the anti-4-1BB×CCR8 antibody described herein is a CCR8 antagonist.

In some implementations, the anti-4-1BB×CCR8 antibodies or antigen-binding fragments thereof can bind to CCR8 (e.g., human CCR8, monkey CCR8, mouse CCR8, and/or chimeric CCR8) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶M, less than 1×10⁻⁷M, less than 1×10⁻⁸ M, less than 1×10⁻⁹ M, or less than 1×10⁻¹⁰ M. In some embodiments, the KD is less than 50 nM, 40 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷M, greater than 1×10⁻⁸ M, greater than 1×10⁻⁹ M, or greater than 1×10⁻¹⁰ M.

Because the anti-4-1BB×CCR8 antibody (e.g., bispecific antibody) binds to both CCR8 and 4-1BB, for cells that express both CCR8 and 4-1BB, the antibody has a higher binding affinity to these cells. Avidity can be used to measure the binding affinity of an antibody to these cells. Avidity is the accumulated strength of multiple affinities of individual non-covalent binding interactions.

In some embodiments, the anti-4-1BB×CCR8 antibody, antigen-binding fragment, or ADC has a tumor growth inhibition rate or percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the anti-4-1BB×CCR8 antibody, antigen-binding fragment, or ADC has a tumor growth inhibition percentage that is less than 60%, 70%, 80%, 90%, 100%, 110%, 120%, 130%, 140%, or 150%. The TGI (%) can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more days after the treatment starts. As used herein, the tumor growth inhibition rate or percentage (TGI %) is calculated using the following formula:

$$TGI ( \% ) = [ 1 - ( T i - T 0 ) / ( Vi ‐ V 0 ) ] \times 100 \%$$

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the anti-4-1BB×CCR8 bispecific antibody, antigen-binding fragment, or ADC reduces tumor volume by 5%, 10%, 15%, 20%, 25%, 30%, 35%, 40%, 45%, 50%, 55%, 60%, 65%, 70%, 75%, 80%, 85%, 90%, 95% or more, compared the tumor size with the administration of PBS or without the administration of the anti-4-1BB×CCR8 antibody, antigen-binding fragment, or ADC. Tumor volume is expressed in mm³ and can be calculated using the formula: “V=(L×W×W)/2, where V is tumor volume, L is tumor length (the longest tumor dimension) and W is tumor width (the longest tumor dimension perpendicular to L).

In some embodiments, the anti-4-1BB×CCR8 antibody, antigen-binding fragment, or ADC has a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the Fc region is human IgG1, human IgG2, human IgG3, or human IgG4. In some embodiments, the Fc region has an enhanced ADCC activity. In some embodiments, the Fc region has an enhanced ADCP activity.

In some embodiments, the anti-4-1BB×CCR8 antibody, antigen-binding fragment, or ADC does not have a functional Fc region. For example, the anti-4-1BB×CCR8 antibodies or antigen-binding fragments thereof are Fab, Fab′, F(ab′)₂, and Fv fragments. In some embodiments, the anti-4-1BB×CCR8 antibodies or antigen-binding fragments thereof as described herein have an Fc region without effector function. In some embodiments, the Fc is a human IgG4 Fc. In some embodiments, the Fc does not have a functional Fc region. For example, the Fc region has LALA mutations (L234A and L235A mutations in EU numbering), or LALA-PG mutations (L234A, L235A, P329G mutations in EU numbering).

In some embodiments, the antibody or antigen-binding fragment thereof comprises a fragment crystallizable region (Fc region). In some embodiments, the bispecific anti-4-1BB×CCR8 antibody described herein can be designed to have an IgG1 subtype structure with the GAALIE mutations (G236A, A330L, and I332E mutations in EU numbering). In some embodiments, the anti-4-1BB×CCR8 antibody described herein can be designed to have an IgG1 Fc region having an Alanine (A) at position 236, a Leucine (L) at position 330, and a Glutamic acid (E) at position 332 in EU numbering.

Some other modifications to the Fc region can be made. For example, a cysteine residue(s) can be introduced into the Fc region, thereby allowing interchain disulfide bond formation in this region. The homodimeric fusion protein thus generated may have any increased half-life in vitro and/or in vivo.

In some embodiments, the IgG4 has S228P mutation (EU numbering). The S228P mutation prevents in vivo and in vitro IgG4 Fab-arm exchange.

In some embodiments, anti-4-1BB×CCR8 antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about +3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the anti-4-1BB×CCR8 antibody can be further engineered to replace the Asparagine at position 297 with Alanine (N297A).

In some embodiments, the bispecific anti-4-1BB×CCR8 antibody described herein has a higher endocytosis rate than the corresponding monoclonal antibodies and/or control bispecific antibodies described herein.

In some embodiments, the methods described herein are designed to make a bispecific anti-4-1BB×CCR8 antibody. Bispecific anti-4-1BB×CCR8 antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

In some embodiments, the anti-4-1BB×CCR8 bispecific antibody, antigen-binding fragment, or ADC promotes antibody-dependent cellular phagocytosis (ADCP), wherein the anti-4-1BB or anti-CCR8 antibody, antigen-binding fragment, or ADC bind to target cells that co-express 4-1BB and/or CCR8 (e.g., CT26 cells or CHO cells), marking them for destruction by phagocytic cells (e.g., macrophages, dendritic cells, or neutrophils). In ADCP, the Fab region of the antibody binds to antigens on the surface of the target cell co-expressing 4-1BB and/or CCR8 (e.g., CT26 cells or CHO cells), while the Fc region interacts with Fc receptors (like CD64 or CD32) on the surface of the phagocytes. Once engaged, this interaction triggers the engulfment and digestion of the target cell co-expressing 4-1BB and/or CCR8 (e.g., CT26 cells or CHO cells) by the phagocyte (e.g., macrophages, dendritic cells, or neutrophils), leading to the clearance of the marked cells (or cell killing). ADCP is a critical mechanism in the immune response and is actively leveraged in therapeutic antibody treatments, particularly in cancer therapy, where antibodies are designed to target and eliminate tumor cells through phagocytosis. ADCP is a key mode of action for many monoclonal antibodies known in arts used in oncology.

In some embodiments, the anti-4-1BB×CCR8 bispecific antibody, antigen-binding fragment, or ADC promotes ADCP of the target cells (e.g., CHO cells) co-expressing hCCR8 and h4-1BB are by effector MO macrophages. In some embodiments, effector cells (e.g., macrophages) to target cells (e.g., CHO cells co-expressing 4-1BB and/or CCR8) ratio or E:T ratio can be any ratio between 1:10 to 100:1 (e.g., between 1:5 to 50:1, between 1:2 to 25:1, or between 1:1 to 10:1).

In some embodiments, knobs-into-holes (KIH) technology can be used, which involves engineering CH3 domains to create either a “knob” or a “hole” in each heavy chain to promote heterodimerization. The KIH technique is described e.g., in Xu, Yiren, et al. “Production of bispecific antibodies in ‘knobs-into-holes’ using a cell-free expression system.” MAbs. Vol. 7. No. 1. Taylor & Francis, 2015, which is incorporated by reference in its entirety. In some embodiments, one heavy chain has a T366W, and/or S354C (knob) substitution (EU numbering), and the other heavy chain has an Y349C, T366S, L368A, and/or Y407V (hole) substitution (EU numbering). In some embodiments, one heavy chain has one or more of the following substitutions Y349C and T366W (EU numbering). The other heavy chain can have one or more the following substitutions E356C, T366S, L368A, and Y407V (EU numbering). Furthermore, a substitution (-ppcpScp-->-ppcpPcp-) can also be introduced at the hinge regions of both substituted IgG.

### Recombinant Vectors

The present disclosure also provides recombinant vectors (e.g., expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of anti-4-1BB×CCR8 antibody polypeptides or fragments thereof by recombinant techniques.

The full length sequence of human CCR8 is known in the art (e.g., NCBI Gene ID: 1237, NCBI Reference Sequence: NM_005201.4). In some embodiments, a His-tagged human CCR8 protein is used as the immunogen.

The full length sequence of human 4-1BB is known in the art (e.g., GenBank Accession No.: U03397.1). In some embodiments, a His-tagged human 4-1BB protein is used as the immunogen.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising a polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y, and Grant et al., Methods Enzymol., 153:516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an anti-4-1BB×CCR8 antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., an anti-4-1BB×CCR8 antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein.

The disclosure also provides a nucleic acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any nucleotide sequence as described herein, and an amino acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any amino acid sequence as described herein.

In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 250, 300, 350, or 400 amino acid residues.

In some embodiments, the amino acid sequence (a) comprises an amino acid sequence; or (b) consists of an amino acid sequence, wherein the amino acid sequence is any one of the sequences as described herein.

In some embodiments, the nucleic acid sequence (a) comprises a nucleic acid sequence; or (b) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The disclosure provides one or more nucleic acid encoding any of the polypeptides as described herein. In some embodiments, the nucleic acid (e.g., cDNA) includes a polynucleotide encoding a polypeptide of a heavy chain as described herein. In some embodiments, the nucleic acid includes a polynucleotide encoding a polypeptide of a light chain as described herein. In some embodiments, the nucleic acid includes a polynucleotide encoding a scFv polypeptide as described herein.

In some embodiments, the vector can have two of the nucleic acids as described herein, wherein the vector encodes the VL region and the VH region that together bind to 4-1BB. In some embodiments, a pair of vectors is provided, wherein each vector comprises one of the nucleic acids as described herein, wherein together the pair of vectors encodes the VL region and the VH region that together bind to 4-1BB.

In some embodiments, the vector includes two of the nucleic acids as described herein, wherein the vector encodes the VL region and the VH region that together bind to CCR8. In some embodiments, a pair of vectors is provided, wherein each vector comprises one of the nucleic acids as described herein, wherein together the pair of vectors encodes the VL region and the VH region that together bind to CCR8.

### Methods of Treatment

The methods described herein include methods for the treatment of disorders associated with cancer. Generally, the methods include administering a therapeutically effective amount of anti-4-1BB×CCR8 antibodies as described herein, to a subject who is in need of, or who has been determined to be in need of, such treatment.

As used in this context, to “treat” means to ameliorate at least one symptom of the disorder associated with cancer. Often, cancer results in death; thus, a treatment can result in an increased life expectancy or survival (e.g., by at least 1, 2, 3, 4, 5, 6, 7, 8, 9, 10, 11, 12 months, or by at least 1, 2, 3, 4, 5, 6, 7, 8, 9, 10 years). Administration of a therapeutically effective amount of an agent described herein for the treatment of a condition associated with cancer will result in decreased number of cancer cells, reduced tumor volume, alleviated symptoms, and/or improved life expectancy or survival rate.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers (e.g., colon carcinoma), renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the cancer is a chemotherapy resistant cancer.

In one aspect, the disclosure also provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of anti-4-1BB×CCR8 antibodies disclosed herein to a subject in need thereof, e.g., a subject having, or identified or diagnosed as having, a cancer, e.g., B cell lymphoma, bladder cancer, breast cancer, cervical cancer, colorectal cancer, gastric cancer, non-small cell lung cancer (NSCLC), mesothelioma, ovarian cancer, pancreatic cancer, prostate cancer, oral cancer, or renal cancer. In some embodiments, the cancer is a solid tumor, lung cancer, head and neck cancer, thyroid cancer, central nervous system (CNS) cancer, liver cancer, or brain cancer.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the anti-4-1BB×CCR8 antibody, anti-4-1BB×CCR8 antigen binding fragment, anti-4-1BB×CCR8 antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an anti-4-1BB×CCR8 antibody or an anti-4-1BB×CCR8 antigen binding fragment is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an anti-4-1BB×CCR8 antibody, anti-4-1BB×CCR8 antigen binding fragment, or anti-4-1BB×CCR8 antibody-drug conjugate may vary, depending on, inter alia, patient history as well as other factors such as the type (and/or dosage) of the agent used.

Effective amounts and schedules for administering the anti-4-1BB×CCR8 antibodies, anti-4-1BB×CCR8 antigen-binding fragments thereof, anti-4-1BB×CCR8 antibody-encoding polynucleotides, and/or compositions disclosed herein may be determined empirically, and making such determinations is within the skill in the art. Those skilled in the art will understand that the dosage that must be administered will vary depending on, for example, the mammal that will receive the anti-4-1BB×CCR8 antibodies, anti-4-1BB×CCR8 antigen-binding fragments thereof, anti-4-1BB×CCR8 antibody-encoding polynucleotides, and/or compositions disclosed herein, the route of administration, the particular type of the agent or compositions disclosed herein used and other drugs being administered to the mammal.

A typical daily dosage of an effective amount of an anti-4-1BB×CCR8 antibody is 0.01 mg/kg to 100 mg/kg. In some embodiments, the dosage can be less than 100 mg/kg, 30 mg/kg, 20 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about or at least 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In some embodiments, the anti-4-1BB×CCR8 bispecific antibody described herein kill tumor cells and/or reduces tumor size (e.g., through the activation of the 4-1BB-related signaling pathway or the recruiting and depletion of CCR8-expressing immune cells, e.g., CCR8-expressing tumor infiltrating Tregs) in a subject receiving the anti-4-1BB×CCR8 bispecific antibody. In some embodiments, about 5%, 10%, 15%, 20%, 25%, 30%, 35%, 40%, 45%, 50%, 55%, 60%, 65%, 70%, 75%, 80%, 85%, 90%, 95% or more of the tumor cells are killed by the administration of the anti-4-1BB×CCR8 bispecific antibody described herein.

In some embodiments, the antitumor effect of the anti-4-1BB×CCR8 bispecific described herein is through the modulation of Treg/Teff cells (e.g., in mice or in humans) to enhance tumor immunity and increase the survival rate.

In some embodiments, the administration of the composition comprising the antibody or antigen-binding fragment thereof that specifically binds to both CCR8 and 4-1BB reduces the tumor volume (tumor size) in the subject. In some embodiments, the tumor volume in a subject receiving the anti-4-1BB×CCR8 bispecific antibody described herein is reduced by at least 5%, 10%, 15%, 20%, 25%, 30%, 35%, 40%, 45%, 50%, 55%, 60%, 65%, 70%, 75%, 80%, 85%, 90%, 95% or more, compared the tumor size without the administration of the anti-4-1BB×CCR8 bispecific antibody. In some embodiments, the tumor size in a subject receiving the anti-4-1BB antibody and anti-CCR8 antibody described herein is reduced by at least 5%, 10%, 15%, 20%, 25%, 30%, 35%, 40%, 45%, 50%, 55%, 60%, 65%, 70%, 75%, 80%, 85%, 90%, 95% or more than the tumor size reduced by the administration of an anti-4-1BB antibody alone or an anti-CCR8 antibody alone.

In some embodiments, the administration of the anti-4-1BB×CCR8 bispecific antibody described herein increases the survival rate of the subject receiving the antibody. In some embodiments, the survival rate is increased by about or at least 5%, 10%, 15%, 20%, 25%, 30%, 35%, 40%, 45%, 50%, 55%, 60%, 65%, 70%, 75%, 80%, 85%, 90%, 95% or more, compared the survival rate of a subject without the administration of the anti-4-1BB×CCR8 bispecific antibody or by the administration of an anti-4-1BB antibody alone or an anti-CCR8 antibody alone.

In some embodiments, one or more additional therapeutic agents can be administered to the subject. The additional therapeutic agent can comprise one or more inhibitors selected from the group consisting of an inhibitor of B-Raf, a 4-1BB inhibitor, an inhibitor of a MEK, an inhibitor of ERK, an inhibitor of K-Ras, an inhibitor of c-Met, an inhibitor of CCR8, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of 4-1BB, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-PD-1 antibody, an anti-PD-L1 antibody, anti-PD-L2 antibody, an anti-LAG-3 antibody, an anti-TIGIT antibody, an anti-BTLA antibody, an anti-CTLA4 antibody, an anti-CD40 antibody, an anti-OX40 antibody, an anti-TIM3 antibody, or an anti-GITR antibody.

### Pharmaceutical Compositions and Routes of Administration

Also provided herein are pharmaceutical compositions that contain at least one (e.g., one, two, three, or four) of the anti-4-1BB×CCR8 antibodies (e.g., bispecific antibodies), anti-4-1BB×CCR8 antigen-binding fragments, or anti-4-1BB×CCR8 antibody-drug conjugates described herein. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant.

Compositions containing one or more of any of the anti-4-1BB×CCR8 antibodies or anti-4-1BB×CCR8 antigen-binding fragments described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the antibodies or antigen-binding fragments, or antibody-drug conjugates (e.g., anti-4-1BB×CCR8 antibodies, anti-4-1BB×CCR8 antigen-binding fragments, or anti-4-1BB×CCR8 antibody-drug conjugates) will be an amount that treats the disease (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies or antigen-binding fragments, or antibody-drug conjugates (e.g., anti-4-1BB×CCR8 antibodies, anti-4-1BB×CCR8 antigen-binding fragments, or anti-4-1BB×CCR8 antibody-drug conjugates) described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments, or antibody-drug conjugates (e.g., anti-4-1BB×CCR8 antibodies, anti-4-1BB×CCR8 antigen-binding fragments, or anti-4-1BB×CCR8 antibody-drug conjugates) described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 1 mg/kg to about 5 mg/kg, about 10 μg/kg to about 0.5 mg/kg; or about 0.1 mg/kg to about 0.5 mg/kg). While these doses cover a broad range, one of ordinary skill in the art will understand that therapeutic agents vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the therapeutic agent in vivo.

The antibodies or antigen-binding fragments, or antibody-drug conjugates (e.g., anti-4-1BB×CCR8 antibodies, anti-4-1BB×CCR8 antigen-binding fragments, or anti-4-1BB×CCR8 antibody-drug conjugates) described herein can be administered one or more times during the course of treatment. For example, the antibodies or antigen-binding fragments, or antibody-drug conjugates (e.g., anti-4-1BB×CCR8 antibodies, anti-4-1BB×CCR8 antigen-binding fragments, or anti-4-1BB×CCR8 antibody-drug conjugates) described herein can be administered 1, 2, 3, 4, 5, 6, 7, 8, 9, 10 or more times. In some embodiments, the antibodies or antigen-binding fragments, or antibody-drug conjugates (e.g., anti-4-1BB×CCR8 antibodies, anti-4-1BB×CCR8 antigen-binding fragments, or anti-4-1BB×CCR8 antibody-drug conjugates) is administered every 1, 2, 3, 4, 5, 6, 7, 8, 9, 10 or more days.

The pharmaceutical compositions can be included in a container, pack, or dispenser together with instructions for administration. The disclosure also provides methods of manufacturing the antibodies or antigen-binding fragments, or antibody-drug conjugates (e.g., anti-4-1BB×CCR8 antibodies, anti-4-1BB×CCR8 antigen-binding fragments, or anti-4-1BB×CCR8 antibody-drug conjugates) for various uses as described herein.

### EXAMPLES

The invention is further described in the following examples, which do not limit the scope of the invention described in the claims.

### Example 1: Molecule Constructions

To generate the anti-CCR8 monoclonal antibody (CCR8 mAb), the VH region was linked with a wild-type mIgG2a Fc, while the VL region was linked with a mouse lambda light constant region. To generate the anti-4-1BB monoclonal antibody (4-1BB mAb), the VH region was linked with a wild-type mIgG2a Fc, while the VL region was linked with a mouse kappa light constant region.

To generate 4-1BB-1×CCR8-1 bispecific antibody (BsAb) and 4-1BB-2×CCR8-1 bispecific antibody (FIG. 1A-1B), the scFv (single-chain fragment variable VH-(G4S)₄-VL) of anti-CCR8 antibody was connected to the N terminal of the anti-4-1BB light chain (LC) through a G4S linker (SEQ ID NO: 86), while the 4-1BB VH was linked with a wild-type mIgG2a Fc.

Three 4-1BB-2×CCR8-2 bispecific antibodies (FIGS. 1C-1E), one 4-1BB-3×CCR8-3 bispecific antibody (FIG. 1F), and one 4-1BB-4×CCR8-4 bispecific antibody (FIG. 1G) were generated as shown in FIG. 1C-1G. All these antibodies comprise a wild-type hIgG1 Fc. Three 4-1BB-2×CCR8-2 bispecific antibodies include 4-1BB-2×CCR8-2 (LC1) BsAb (FIG. 1C), 4-1BB-2×CCR8-2 (LC2) BsAb (FIG. 1D), and 4-1BB-2×CCR8-2 (HC) BsAb (FIG. 1E).

Fragments were synthesized (IDT) and assembled (NEB NEBuilder® HiFi DNA Assembly) into pcDNA3.4 or pcDNA3.4 carrying desired fragments as described above. The assembled plasmid was transformed to Competent E. coli (NEB® 5-alpha). Clones with the correct sequence, confirmed by sequencing results (Elim Biopharm), were further cultured with lysogeny broth (LB) containing Carbenicillin (100 μg/ml) for plasmid amplification, followed by plasmid purification (QIAGEN Plasmid Plus Kits). Plasmids were eluted in Nuclease-free H₂O (Sigma) and stored at −80° C.

### Example 2: Transfection and Purification of Antibodies

All antibody light chain and heavy chain coding sequences were generated by direct DNA synthesis and cloned into a mammalian expression vector. The cloned sequences were verified by Sanger Sequencing. Chinese hamster ovary (CHO) cells were grown in CHOgro medium (Mirus) complemented with 4 mM L-Glutamine and 0.33% Poloxamer-188 and diluted to 4×10⁶ cells/ml with fresh cell culture media at the time of transfection. Cells were transfected by lipofection with 1 μg of plasmid DNA per 4×10⁶ cells. Transfected cells were kept in incubator with shaking at 125 rpm in 5% CO₂ and 70% humidity environment at 32° C.

Protein expression titers were measured every 5 days. Cell supernatant was collected by centrifugation after 10-14 days post-transfection and filtered through a 0.22 μm PES membrane filter. Expressed antibodies were purified by Protein A chromatography (TOYOPEARL® AF-rProtein A HC-650M resin, Tosoh) and aggregates were removed by anion exchange resin (TOYOPEARL NH2-750F, Tosoh).

Purified antibodies were analyzed by HPLC (Dionex UltiMate® 3000-RS UHPLC, ThermoFisher) controlled by Chromeleon® software. A size exclusion column TSKgel UP-SW3000, 2 μm, 4.6 mm ID×15 cm (Tosoh) was used to resolve antibodies purity and aggregation.

All purified antibodies had 97% or greater monomer purity, were sterilized through 0.22 μm PES membrane filter, and used for binding and functional assays.

### Example 3: Anti-Tumor Efficacy of 4-1BB×CCR8 Bispecific Antibody in CT26 Colon Carcinoma Model (Study 1)

The anti-tumor effects of 4-1BB×CCR8 bispecific antibodies were examined against CT26 colon carcinoma cells using BalB/c mice. Female BalB/c mice, 6-9 weeks old, were obtained from Vital River Laboratories Research Models and Services (Guangdong, China) and used in the experiments. All experiments were approved by the Institutional Animal Care and Use Committee (IACUC). As shown in FIG. 2, CT26 colon carcinoma cells in logarithmic growth phase were injected subcutaneously (5×10⁵ cells) in the right rear flank of the mice on day 0 in 0.1 ml of PBS for tumor development. Mice were randomized and assigned to treatment groups when the mean tumor size reached approximately 80-120 mm³ on day 7. Mice were treated with 4-1BB-1×CCR8-1 bispecific antibody, CCR8-1 mAb and 4-1BB-1 mAb on days 7, 10, 14 and 18. The 1^{st}, 2^{nd}, and 3^{rd} doses were 1 mg/kg of each mAb and 1.33 mg/kg BsAb whereas the 4th dose was 10 mg/kg for mAbs and BsAb. Control group's mice were treated with Phosphate buffer saline (PBS). The 1^{st} dose was given intravenously while the 2^{nd}, 3^{rd}, and 4^{th} doses were administered via intraperitoneal injections. The tumors were measured three times per week in two dimensions using a caliper, and the volume were expressed in mm³ using the formula: V=(L×W×W)/2, where V is tumor volume, L is tumor length (the longest tumor dimension), and W is tumor width (the longest tumor dimension perpendicular to L). Graphing and statistical analysis was performed using GraphPad Prism program, two-way ANOVA with Dunnett multiple comparisons test for tumor growth.

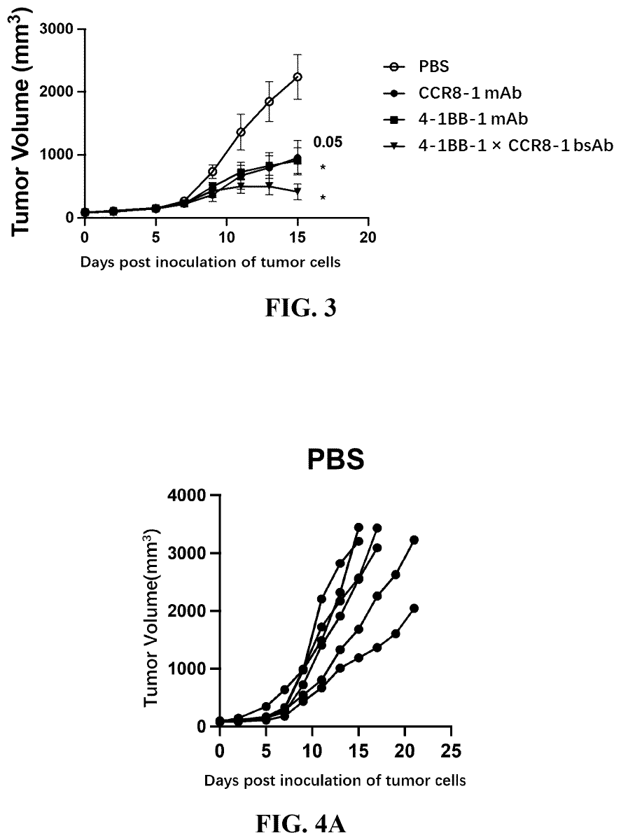

In the comparison of a mean tumor volume between the single mAb administration groups (e.g., single-agent CCR8-1 mAb or single-agent 4-1BB-1 mAb administration groups) and 4-1BB-1×CCR8-1 bispecific antibody administration group, the mean tumor volume was significantly smaller in the 4-1BB-1×CCR8-1 bispecific antibody administration group compared with the single mAb administration groups (FIG. 3).

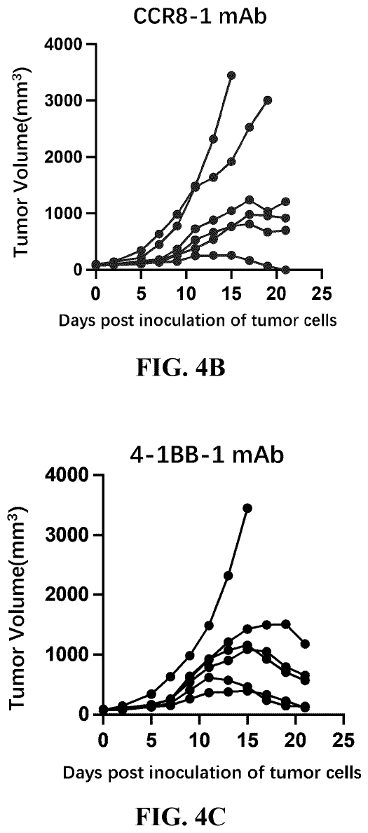

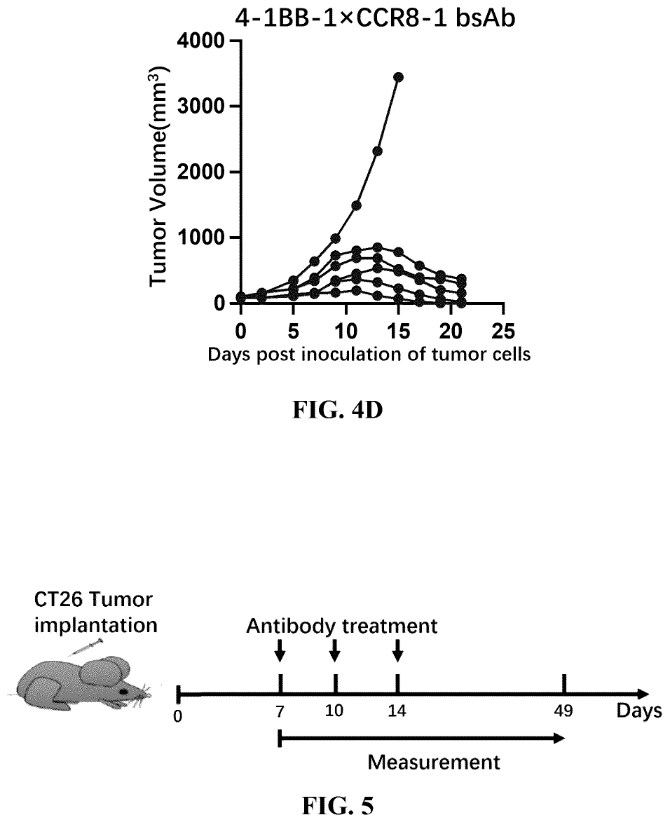

A complete remission rate of the tumors at days was also compared. In the end of the study at Day 28, the treatment with 4-1BB-1×CCR8-1 bispecific antibody led to higher number of tumor-free mice (2 tumor free mice out of 6) compared to either CCR8-1 mAb (1 tumor free mice out of 6) or 4-1BB-1 mAb (no tumor free mice out of 6) treatment alone (FIGS. 4A-4D).

These results demonstrated that the 4-1BB×CCR8 bispecific antibody is highly effective in inhibiting tumor growth as compared with other single mAbs in this CT26 mouse model. 4-1BB-1×CCR8-1 bispecific antibody induced stronger anti-tumor effect in form of tumor volume inhibition and tumor free mice compared to either monoclonal anti-CCR8 or anti-4-1BB antibodies alone.

These results suggest that, demonstrated by the antitumor effects of the anti-4-1BB×CCR8 bispecific antibody administered to mice, Treg/Teff cells can be modulated in humans to enhance anti-tumor immunity and increase the survival rate.

The major concern of 4-1BB antibody therapy is dose-limiting liver toxicity, as observed in urelumab's clinical development. Most common adverse events were elevated levels of alanine transaminase (ALT) and aspartate aminotransferase (AST). Blood samples were collected from CT26 cell-implanted mice for ALT and AST measurements after the treatment of 4-1BB-1×CCR8-1 bispecific antibody administration. No significant elevation of ALT and AST was observed, suggesting little risk for liver toxicity commonly induced by 4-1BB×CCR8 bispecific antibody treatment.

### Example 4: Anti-Tumor Efficacy of 4-1BB×CCR8 Bispecific Antibody in CT26 Colon Carcinoma Model (Study 2)

The anti-tumor effects of 4-1BB×CCR8 bispecific antibodies were examined against CT26 colon carcinoma model using BalB/c mice. Female BalB/c mice, 6-9 weeks old, were obtained from Vital River Laboratories Research Models and Services (Guangdong, China), and used in the experiments. All experiments were approved by the Institutional Animal Care and Use Committee (IACUC). As shown in FIG. 5, CT26 colon carcinoma cells in logarithmic growth phase were injected subcutaneously (5×10⁵ cells) into the right rear flank of the mice on day 0 in 0.1 ml of PBS for tumor development. Mice were randomized and assigned to the treatment groups when the mean tumor size reached approximately 80-120 mm³ on day 7. Mice were treated with 4-1BB-1×CCR8-1 bispecific antibody, CCR8-1 mAb, or 4-1BB-1 mAb on days 7, 10 and 14. Dose titration was performed with 1 mg/kg or 3 mg/kg of each monoclonal mAb, and 1.33 mg/kg or 4 mg/kg of bispecific antibodies. Control group mice were treated with Phosphate buffer saline (PBS). The 1^{st} dose was given intravenously, while the 2^{nd} and 3^{rd} doses were administered via intraperitoneal injections. The tumors were measured three times per week in two dimensions using a caliper, and the volume were expressed in mm³ using the formula: V=(L×W×W)/2, where V is tumor volume, L is tumor length (the longest tumor dimension) and W is tumor width (the longest tumor dimension perpendicular to L). Graphing and statistical analysis was performed using GraphPad Prism program. Two-way ANOVA with Dunnett multiple comparisons test was used for analyzing tumor growth.

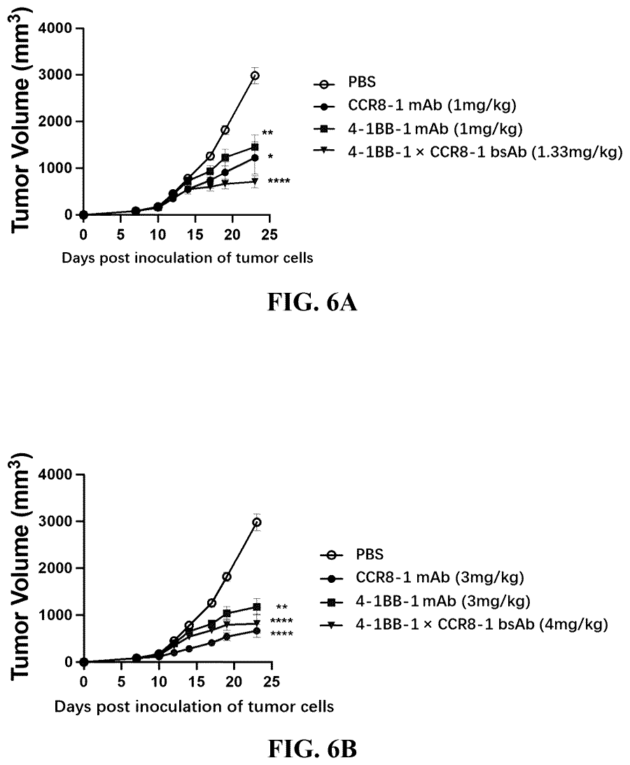

In the comparison of a mean tumor volume between the single mAb administration groups and 4-1BB-1×CCR8-1 bispecific antibody administration group, the mean tumor volume was significantly smaller in the 4-1BB-1×CCR8-1 bispecific antibody administration group compared with the single mAb administration groups at day 17, 19 and 23 (FIGS. 6A-6B).

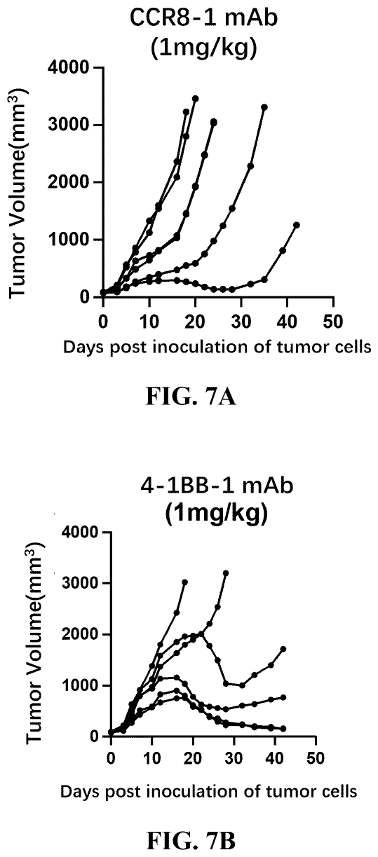

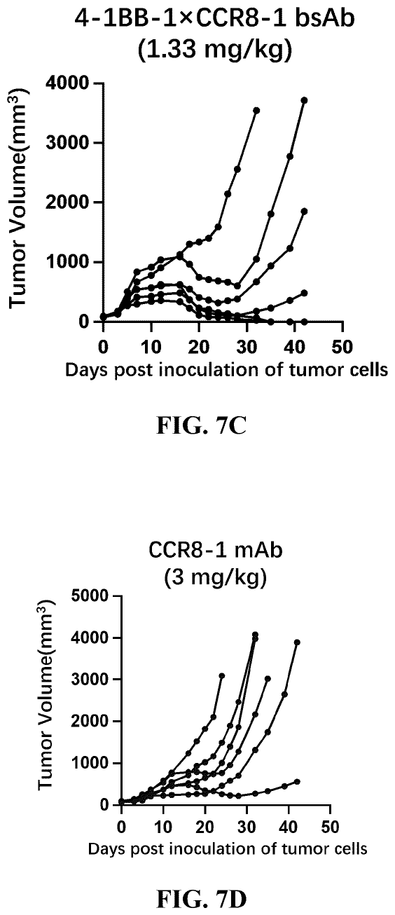

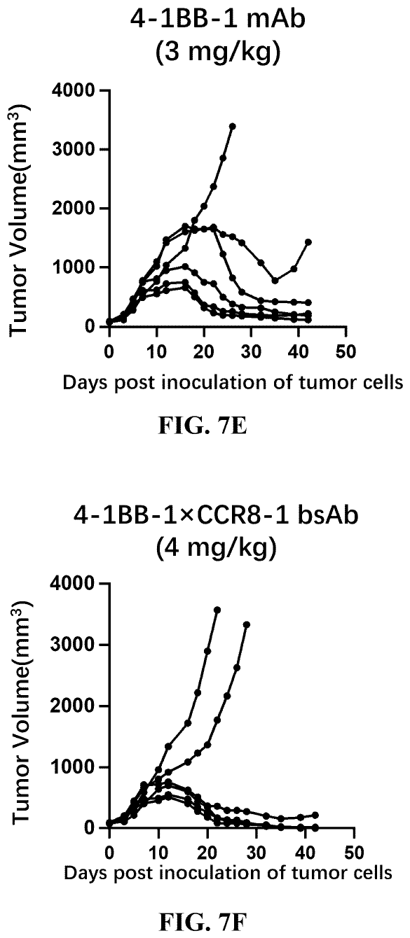

A complete remission rate of the tumors at days was also compared in the end of the study at day 49. For 1 mg/kg of each single mAb or 1.33 mg/kg bispecific antibody dose, the treatment with 4-1BB-1×CCR8-1 antibody led to higher number of tumor free mice (2 tumor free mice out of 6) compared to either CCR8-1 mAb (no tumor free mice out of 6) and 4-1BB-1 mAb (no tumor free mice out of 6) treatment alone (FIGS. 7A-7C). For 3 mg/kg of each single mAb and 4 mg/kg bispecific antibody dose, the treatment with 4-1BB-1×CCR8-1 antibody led to higher number of tumor free mice (3 tumor free mice out of 6) compared to either CCR8-1 mAb (no tumor free mice out of 6) or 4-1BB-1 mAb (no tumor free mice out of 6) treatment alone (FIGS. 7D-7F). Moreover, 4-1BB-1×CCR8-1 bispecific antibody administration groups required shorter time to tumor regression as compared with either monoclonal CCR8-1 or 4-1BB-1 mAb alone.

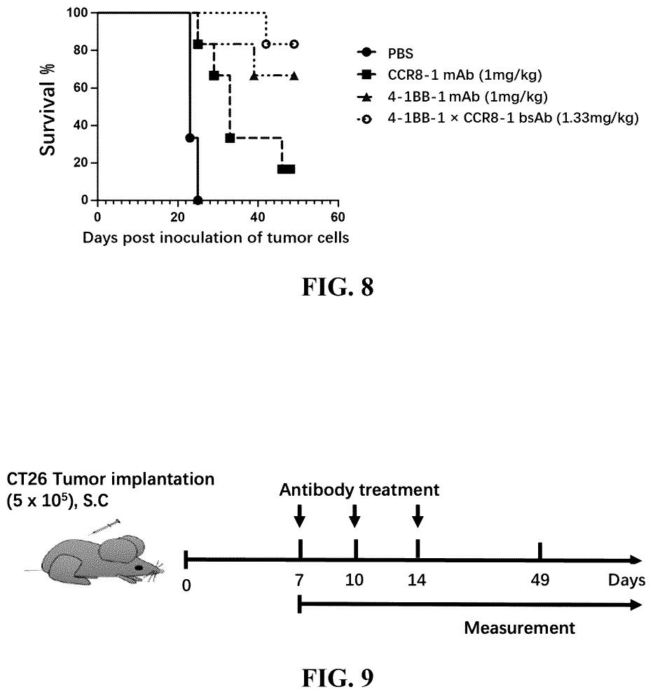

Percent survival rates during the treatment were also calculated. The 4-1BB-1×CCR8-1 bispecific antibody treatment led to increased overall percent survival among mice, compared to the treatment with either CCR8-1 or 4-1BB-1 monoclonal antibodies alone (FIG. 8).

These results demonstrated that the 4-1BB×CCR8 bispecific antibody administration groups require a short time to tumor regression, and exhibit a strong regressing effect and high survival percentage as compared with the treatment with a monoclonal CCR8 or 4-1BB mAb alone in CT26 mouse model.

The major concern of 4-1BB antibody therapy is dose-limiting liver toxicity, as observed in urelumab's clinical development. Most common adverse events were elevated levels of alanine transaminase (ALT) and aspartate aminotransferase (AST). Blood samples were collected for ALT and AST measurements from CT26 mice after the treatment of 4-1BB-1×CCR8-1 bispecific antibody administration. No significant elevation of ALT and AST was observed, suggesting little risk for liver toxicity commonly induced by 4-1BB×CCR8 bispecific antibody treatment.

### Example 5: Anti-Tumor Efficacy of 4-1BB×CCR8 Bispecific Antibody in CT26 Colon Carcinoma Model (Study 3)

The anti-tumor effects of 4-1BB×CCR8 bispecific antibody were examined against CT26 colon carcinoma model using BalB/c mice. Female BalB/c mice, 6-9 weeks old, were obtained from Vital River Laboratories Research Models and Services (Guangdong, China) and used in the experiments. All experiments were approved by the Institutional Animal Care and Use Committee (IACUC). As shown in FIG. 9. CT26 colon carcinoma cells in logarithmic growth phase were injected subcutaneously (5×10⁵ cells) into the right rear flank of the mice on day 0 in 0.1 ml of PBS for tumor development. Mice were randomized and assigned to the treatment groups when mean tumor size reached approximately 80-120 mm³ on day 7. Mice were treated with 4-1BB-2×CCR8-1 bispecific antibody, CCR8-1 mAb, or 4-1BB-2 mAb on days 7, 10 and 14. Dose titration was performed with 1 mg/kg of each monoclonal mAb, and 1.33 mg/kg of bispecific antibodies. Control group mice were treated with Phosphate buffer saline (PBS). The 1^{st} dose was given intravenously, while the 2^{nd} and 3^{rd} doses were administered via intraperitoneal injections. The tumors were measured three times per week in two dimensions using a caliper, and the volume were expressed in mm³ using the formula: V=(L×W×W)/2, where V is tumor volume, L is tumor length (the longest tumor dimension) and W is tumor width (the longest tumor dimension perpendicular to L). Graphing and statistical analysis was performed using GraphPad Prism program, and two-way ANOVA with Dunnett multiple comparisons test was used to analyze tumor growth.

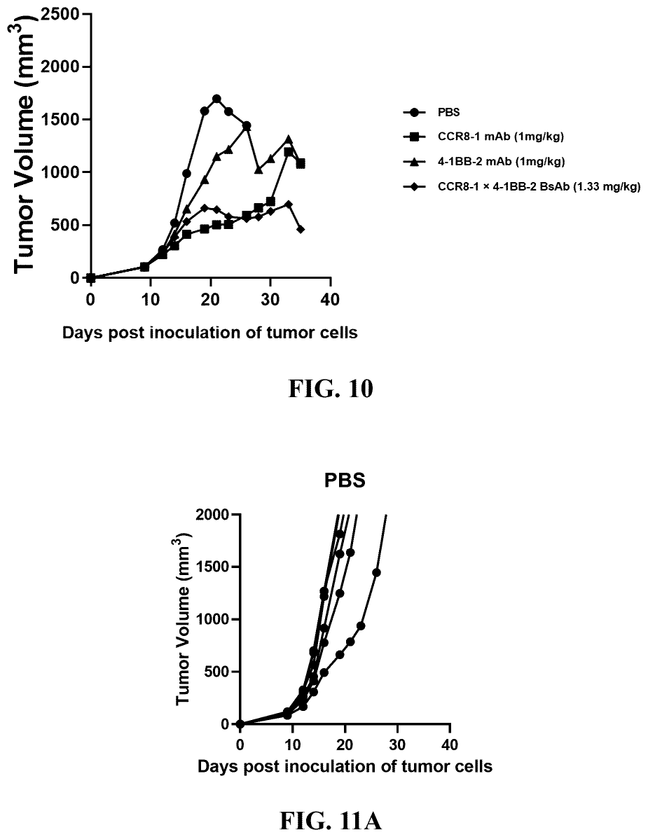

In the comparison of a mean tumor volume between the single mAb administration groups and 4-1BB-2×CCR8-1 bispecific antibody administration group, the mean tumor volume was significantly smaller in the 4-1BB-2×CCR8-1 bispecific antibody administration group compared with the each of the single mAb administration groups after Day 30 (FIG. 10).

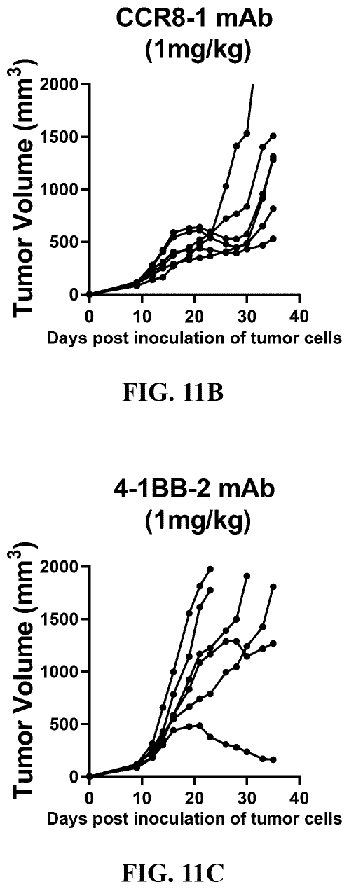

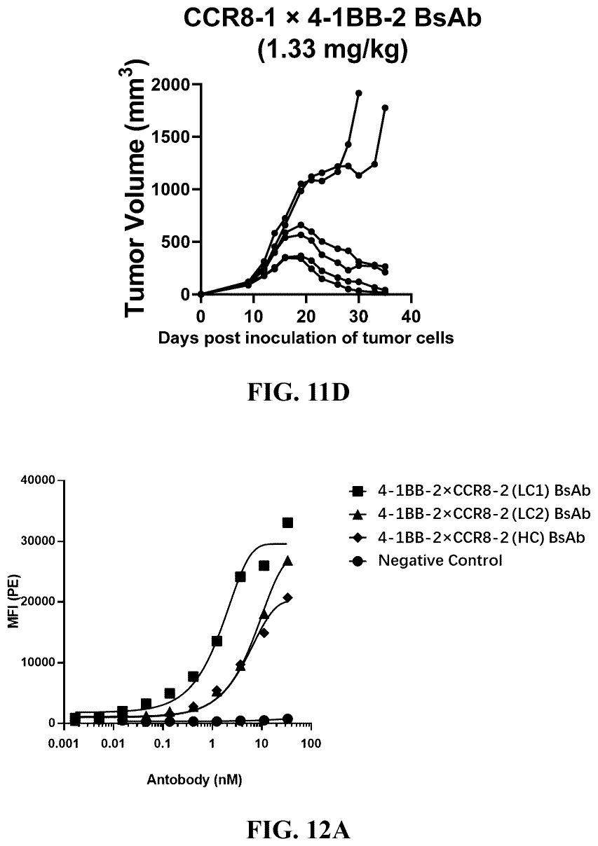

A complete remission rate of the tumors at days was also compared in the end of the study at day 49. For 1 mg/kg of each single mAb or 1.33 mg/kg bispecific antibody dose, the treatment with 4-1BB-2×CCR8-1 bispecific antibody led to higher number of tumor free mice (2 tumor free mice out of 6) compared to either CCR8-1 mAb (no tumor free mice out of 6) or 4-1BB-2 mAb (no tumor free mice out of 6) treatment alone (FIGS. 11A-11D).

These results demonstrated that the 4-1BB×CCR8 bispecific antibody administration groups require a short time to tumor regression, and exhibit a strong regressing effect and high survival percentage as compared with the treatment with a monoclonal CCR8 or 4-1BB mAb alone in CT26 mouse model.

The major concern of 4-1BB antibody therapy is dose-limiting liver toxicity, as observed in urelumab's clinical development. Most common adverse events were elevated levels of alanine transaminase (ALT) and aspartate aminotransferase (AST). Blood samples were collected for ALT and AST measurements from CT26 mice after the treatment of 4-1BB-2×CCR8-1 bispecific antibody administration. No significant elevation of ALT and AST was observed, suggesting little risk for liver toxicity commonly induced by 4-1BB×CCR8 bispecific antibody treatment.

### Example 6. Binding of 4-1BB×CCR8 Bispecific Antibodies to CHO Cells Expressing Human 4-1BB

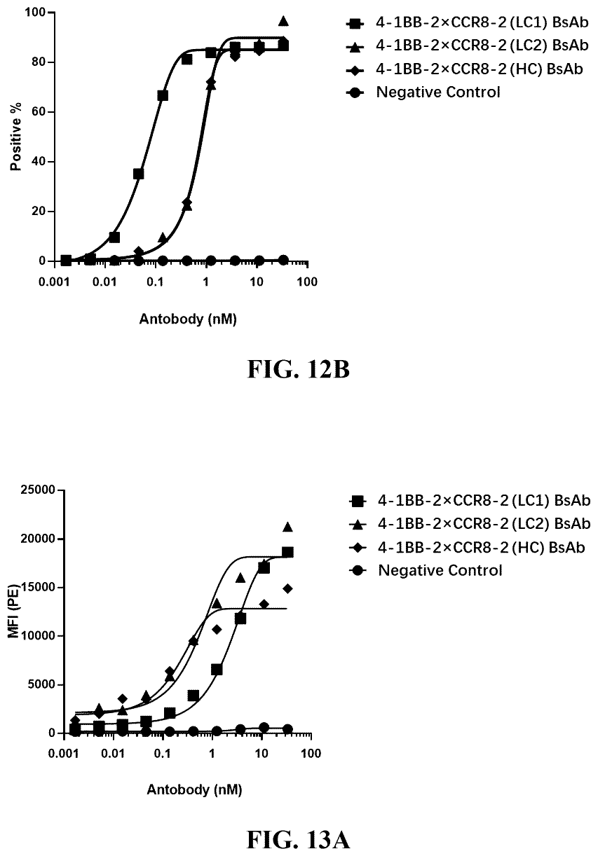

Binding abilities of 4-1BB×CCR8 bispecific antibodies (BsAb) to CHO cells expressing human 4-1BB were evaluated. About 50,000 CHO cells expressing human 4-1BB were incubated with serially diluted antibodies (4-1BB-2×CCR8-2 (LC1) BsAb, 4-1BB-2×CCR8-2 (LC2) BsAb, and 4-1BB-2×CCR8-2 (HC) BsAb) in FACS buffer (PBS+0.5% BSA) for 30 minutes on ice. After incubation, cells were washed twice and detected with anti-human IgG PE secondary antibody (Jackson ImmunoResearch, Cat. #: 109-116-170). For analysis, the mean fluorescence intensity (MFI of PE) of each antibody was determined and plotted using GraphPad Prism software (Version 9.4.1; GraphPad Software Inc.). As shown in FIGS. 12A-12B, all the three 4-1BB×CCR8 bispecific antibodies bind to CHO cells expressing human 4-1BB very well.

### Example 7. Binding of 4-1BB×CCR8 Bispecific Antibodies to CHO Cells Expressing Human CCR8

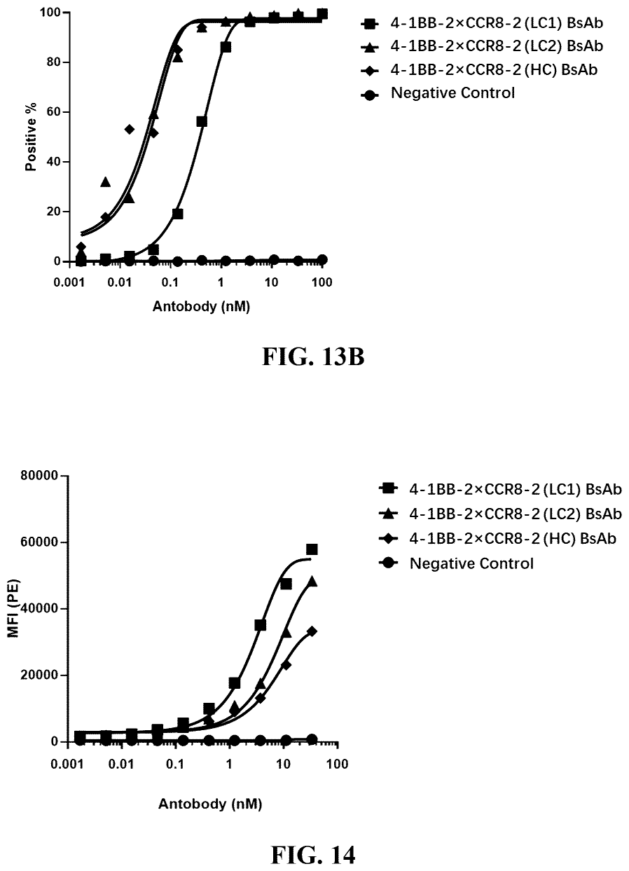

Binding abilities of 4-1BB×CCR8 bispecific antibodies to CHO cells expressing human CCR8 were evaluated. About 50,000 CHO cells expressing human CCR8 were incubated with serially diluted antibodies (4-1BB-2×CCR8-2 (LC1) BsAb, 4-1BB-2×CCR8-2 (LC2) BsAb and 4-1BB-2×CCR8-2 (HC) BsAb) in FACS buffer (PBS+0.5% BSA) for 30 minutes on ice. After incubation, cells were washed twice and detected with anti-human The mean fluorescence intensity (MFI of PE) of each antibody was determined and plotted using GraphPad Prism software (Version 9.4.1; GraphPad Software Inc.). As shown in FIGS. 13A-13B, all the three 4-1BB×CCR8 bispecific antibodies bind to CHO cells expressing human CCR8 very well.

### Example 8. Binding of 4-1BB×CCR8 Bispecific Antibodies to CHO Cells Co-Expressing Human CCR8 and 4-1BB

Binding abilities of 4-1BB×CCR8 bispecific antibodies to CHO cells co-expressing human CCR8 and 4-1BB were evaluated. About 50,000 CHO cells co-expressing human CCR8 and 4-1BB were incubated with serially diluted antibodies (4-1BB-2×CCR8-2 (LC1) BsAb, 4-1BB-2×CCR8-2 (LC2) BsAb, and 4-1BB-2×CCR8-2 (HC) BsAb) in FACS buffer (PBS+0.5% BSA) for 30 minutes on ice. After incubation, cells were washed twice and detected with anti-human IgG PE secondary antibody (Jackson ImmunoResearch, Cat. #: 109-116-170). For analysis, the mean fluorescence intensity (MFI of PE) of each antibody was determined and plotted using GraphPad Prism software (Version 9.4.1; GraphPad Software Inc.). As shown in FIG. 14, all the three 4-1BB×CCR8 bispecific antibodies bind to CHO cells co-expressing CCR8 and 4-1BB very well.

### Example 9. 4-1BB×CCR8 Bispecific Antibodies Mediated the Cell Killing of CHO Cells Co-Expressing Human CCR8 and 4-1BB by PBMC

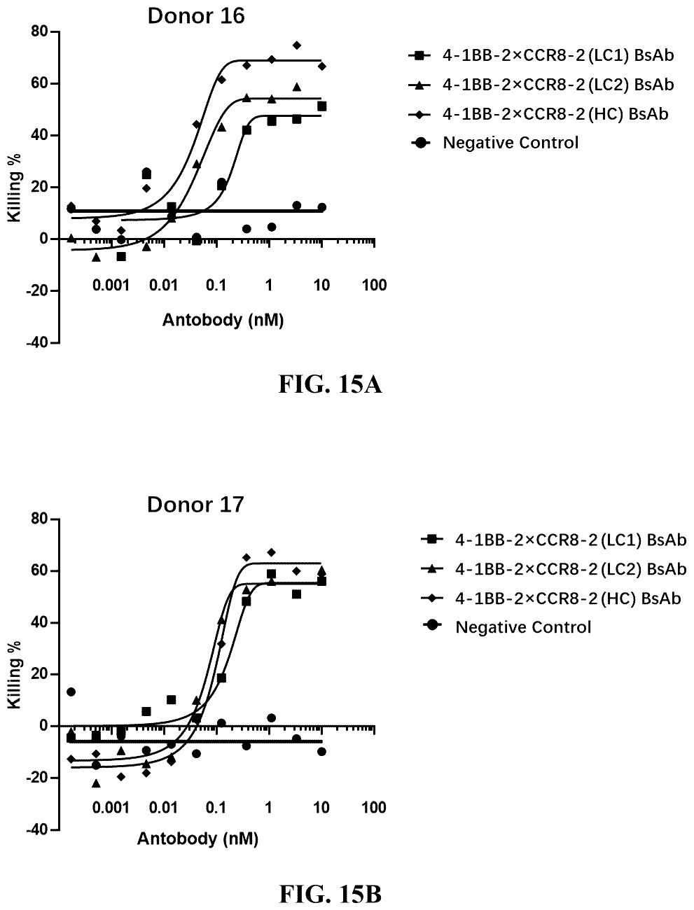

The killing efficacy of the target CHO cells co-expressing hCCR8 and h4-1BB in the presence of different formats of 4-1BB×CCR8 bispecific antibodies by effector PBMC cells were assessed. PBMCs were isolated from the healthy human blood sample of human donor #16 and #17 by ficoll method. CHO cells were labeled with CFSE (ThermoFisher, Cat. #: C34554). About 100,000 PBMCs were incubated with 10,000 CFSE+ CHO to maintain effector to target (E:T) ratios of 10:1 in the presence of serially diluted antibodies (4-1BB-2×CCR8-2 (LC1) BsAb, 4-1BB-2×CCR8-2 (LC2) BsAb, and 4-1BB-2×CCR8-2 (HC) BsAb) in complete RPMI media (supplemented with 10% heat-inactivated FBS and 5% penicillin/streptomycin) for 24 hours at 37° C. After incubation, the cells were washed with FACS buffer (buffer used in fluorescence-activated cell sorting) twice and stained with 50 μl of Live dead Near-IR (Thermo Fisher, Cat. #: L10119) at 1:2000 dilution in PBS at room temperature for 10 mins, and followed with two washes. Cells were resuspended in 100 μl of FACS buffer and analyzed with a Cytek® cytometer. CHO cells were gated as CFSE+ by FACS, and the absolute cell count of CFSE+ cells was obtained by collecting a fixed volume for all treatment conditions. Percentage of target cell killing was calculated as: (absolute number of CFSE+ CHO cell number in non-treatment group-absolute number of CFSE+ Live deadNear-IR− CHO cell number in treatment group)/absolute number of CFSE+ CHO cell number in non-treatment group×100. As shown in FIGS. 15A-15B, all the three 4-1BB×CCR8 bispecific antibodies with different formats efficiently enhanced the cell killing of CHO cells co-expressing hCCR8 and h4-1BB.

### Example 10. Additional Exemplary 4-1BB×CCR8 BsAbs

Additional 4-1BB×CCR8 BsAb (4-1BB-3×CCR8-3 BsAb, 4-1BB-4×CCR8-4 BsAb) are also tested, each BcsAb shows higher anti-tumor effects, lower liver toxicity, and lower E:T ratio, compared to CCR8 mAb or 4-1BB mAb alone.

These results suggest that, as demonstrated by the antitumor effects of the anti-4-1BB×CCR8 bispecific antibody administered to mice, Treg/Teff cells can be modulated to enhance tumor immunity and increase the survival rate in humans.

TABLE 1
Amino acid sequences described in the present disclosure

| SEQ | | |
|---|---|---|
| ID | | |
| NO. | description | sequence |
| 1 | 4-1BB-1 mAb | SDGVH |
| | HCDR1 | |
| 2 | 4-1BB-1 mAb | IIYYDGGTDYNSAIKS |
| | HCDR2 | |
| 3 | 4-1BB-1 mAb | IHFDY |
| | HCDR3 | |
| 4 | 4-1BB-1 mAb | RASSSVSYMY |
| | LCDR1 | |
| 5 | 4-1BB-1 mAb | DTSKLAS |
| | LCDR2 | |
| 6 | 4-1BB-1 mAb | QQWSSTPLT |
| | LCDR3 | |
| 7 | 4-1BB-1 mAb | QVQLKEAGPGLVQPTQTLSLTCTVSGFSLTSDGVHWIRQPPGKGLEWM |
| | VH | GIIYYDGGTDYNSAIKSRLSISRDTSKSQVFLKINSLQTDDTAMYYCARI |
| | | HFDYWGQGVMVTVSS |
| 8 | 4-1BB-1 mAb | DIVLTQSPTTIAASPGEKVTITCRASSSVSYMYWYQQKSGASPKLWIYD |
| | VL | TSKLASGVPNRFSGSGSGTSYSLAINTMETEDAATYYCQQWSSTPLTFG |
| | | SGTKLEIKR |
| 9 | 4-1BB-1 mAb | QVQLKEAGPGLVQPTQTLSLTCTVSGFSLTSDGVHWIRQPPGKGLEWM |
| | heavy chain | GIIYYDGGTDYNSAIKSRLSISRDTSKSQVFLKINSLQTDDTAMYYCARI |
| | | HFDYWGQGVMVTVSSAKTTAPSVYPLAPVCGDTTGSSVTLGCLVKGY |
| | | FPEPVTLTWNSGSLSSGVHTFPAVLQSDLYTLSSSVTVTSSTWPSQSITC |
| | | NVAHPASSTKVDKKIEPRGPTIKPCPPCKCPAPNLLGGPSVFIFPPKIKDV |
| | | LMISLSPIVTCVVVDVSEDDPDVQISWFVNNVEVHTAQTQTHREDYNS |
| | | TLRVVSALPIQHQDWMSGKEFKCKVNNKDLPAPIERTISKPKGSVRAPQ |
| | | VYVLPPPEEEMTKKQVTLTCMVTDFMPEDIYVEWTNNGKTELNYKNT |
| | | EPVLDSDGSYFMYSKLRVEKKNWVERNSYSCSVVHEGLHNHHTTKSF |
| | | SRTPGK |
| 10 | 4-1BB-1 mAb | DIVLTQSPTTIAASPGEKVTITCRASSSVSYMYWYQQKSGASPKLWIYD |
| | light chain | TSKLASGVPNRFSGSGSGTSYSLAINTMETEDAATYYCQQWSSTPLTFG |
| | | SGTKLEIKRADAAPTVSIFPPSSEQLTSGGASVVCFLNNFYPKDINVKWK |
| | | IDGSERQNGVLNSWTDQDSKDSTYSMSSTLTLTKDEYERHNSYTCEAT |
| | | HKTSTSPIVKSFNRNEC |
| 11 | CCR8-1 mAb | DYGVH |
| | HCDR1 | |
| 12 | CCR8-1 mAb | GVSWNGSRTHYADSVKG |
| | HCDR2 | |
| 13 | CCR8-1 mAb | RGA |
| | HCDR3 | |
| 14 | CCR8-1 mAb | SGSSFNIGSHFVY |
| | LCDR1 | |
| 15 | CCR8-1 mAb | KNNQRPS |
| | LCDR2 | |
| 16 | CCR8-1 mAb | AAWDDSLNGPV |
| | LCDR3 | |
| 17 | CCR8-1 mAb | EVQLLESGGGLVQPGGSLRLSCAASGFTFSDYGVHWVRQAPGKGLEW |
| | VH | VSGVSWNGSRTHYADSVKGRFTISRDNSKNTLYLQMNSLRAEDTAVY |
| | | YCVTRGAWGQGTLVTVSS |
| 18 | CCR8-1 mAb | QSVLTQPPSASGTPGQRVTISCSGSSFNIGSHFVYWYQQLPGTAPKLLIY |
| | VL | KNNQRPSGVPDRFSGSKSGTSASLAISGLRSEDEADYYCAAWDDSLNG |
| | | PVFGGGTKLTVL |
| 19 | CCR8-1 mAb | EVQLLESGGGLVQPGGSLRLSCAASGFTFSDYGVHWVRQAPGKGLEW |
| | heavy chain | VSGVSWNGSRTHYADSVKGRFTISRDNSKNTLYLQMNSLRAEDTAVY |
| | | YCVTRGAWGQGTLVTVSSAKTTAPSVYPLAPVCGDTTGSSVTLGCLVK |
| | | GYFPEPVTLTWNSGSLSSGVHTFPAVLQSDLYTLSSSVTVTSSTWPSQSI |
| | | TCNVAHPASSTKVDKKIEPRGPTIKPCPPCKCPAPNLLGGPSVFIFPPKIK |
| | | DVLMISLSPIVTCVVVDVSEDDPDVQISWFVNNVEVHTAQTQTHREDY |
| | | NSTLRVVSALPIQHQDWMSGKEFKCKVNNKDLPAPIERTISKPKGSVRA |
| | | PQVYVLPPPEEEMTKKQVTLTCMVTDFMPEDIYVEWTNNGKTELNYK |
| | | NTEPVLDSDGSYFMYSKLRVEKKNWVERNSYSCSVVHEGLHNHHTTK |
| | | SFSRTPGK |
| 20 | CCR8-1 mAb | QSVLTQPPSASGTPGQRVTISCSGSSFNIGSHFVYWYQQLPGTAPKLLIY |
| | light chain | KNNQRPSGVPDRFSGSKSGTSASLAISGLRSEDEADYYCAAWDDSLNG |
| | | PVFGGGTKLTVLGQPKSSPSVTLFPPSSEELETNKATLVCTITDFYPGVV |
| | | TVDWKVDGTPVTQGMETTQPSKQSNNKYMASSYLTLTARAWERHSS |
| | | YSCQVTHEGHTVEKSLSRADCS |
| 21 | 4-1BB-1 × | QVQLKEAGPGLVQPTQTLSLTCTVSGFSLTSDGVHWIRQPPGKGLEWM |
| | CCR8-1 BsAb | GIIYYDGGTDYNSAIKSRLSISRDTSKSQVFLKINSLQTDDTAMYYCARI |
| | heavy chain | HFDYWGQGVMVTVSSAKTTAPSVYPLAPVCGDTTGSSVTLGCLVKGY |
| | | FPEPVTLTWNSGSLSSGVHTFPAVLQSDLYTLSSSVTVTSSTWPSQSITC |
| | | NVAHPASSTKVDKKIEPRGPTIKPCPPCKCPAPNLLGGPSVFIFPPKIKDV |
| | | LMISLSPIVTCVVVDVSEDDPDVQISWFVNNVEVHTAQTQTHREDYNS |
| | | TLRVVSALPIQHQDWMSGKEFKCKVNNKDLPAPIERTISKPKGSVRAPQ |
| | | VYVLPPPEEEMTKKQVTLTCMVTDFMPEDIYVEWTNNGKTELNYKNT |
| | | EPVLDSDGSYFMYSKLRVEKKNWVERNSYSCSVVHEGLHNHHTTKSF |
| | | SRTPGK |
| 22 | 4-1BB-1 × | EVQLLESGGGLVQPGGSLRLSCAASGFTFSDYGVHWVRQAPGKGLEW |
| | CCR8-1 BsAb | VSGVSWNGSRTHYADSVKGRFTISRDNSKNTLYLQMNSLRAEDTAVY |
| | light chain | YCVTRGAWGQGTLVTVSSGGGGSGGGGSGGGGSGGGGSQSVLTQPPS |
| | | ASGTPGQRVTISCSGSSFNIGSHFVYWYQQLPGTAPKLLIYKNNQRPSG |
| | | VPDRFSGSKSGTSASLAISGLRSEDEADYYCAAWDDSLNGPVFGGGTK |
| | | LTVLGGGGSGGGGSGGGGSDIVLTQSPTTIAASPGEKVTITCRASSSVSY |
| | | MYWYQQKSGASPKLWIYDTSKLASGVPNRFSGSGSGTSYSLAINTMET |
| | | EDAATYYCQQWSSTPLTFGSGTKLEIKRADAAPTVSIFPPSSEQLTSGGA |
| | | SVVCFLNNFYPKDINVKWKIDGSERQNGVLNSWTDQDSKDSTYSMSST |
| | | LTLTKDEYERHNSYTCEATHKTSTSPIVKSFNRNEC |
| 23 | Linker 1 | GGGGSGGGGSGGGGSGGGGS |
| 24 | 4-1BB-2 mAb | DYWVS |
| | HCDR1 | |
| 25 | 4-1BB-2 mAb | EIYPNSGATNFNGKFRG |
| | HCDR2 | |
| 26 | 4-1BB-2 mAb | EYTRDWFAY |
| | HCDR3 | |
| 27 | 4-1BB-2 mAb | RSSQSLLDSDGNTYLY |
| | LCDR1 | |
| 28 | 4-1BB-2 mAb | LVSNLGS |
| | LCDR2 | |
| 29 | 4-1BB-2 mAb | MQPTHAPYT |
| | LCDR3 | |
| 30 | 4-1BB-2 mAb | EVQLVQSGAEVKKPGSSVKVSCKASDYTFNDYWVSWVRQAPGQGLE |
| | VH | WIGEIYPNSGATNFNGKFRGRATLTVDNSASTAYMELSSLRSEDTAVY |
| | | YCTREYTRDWFAYWGQGTLVTVSS |
| 31 | 4-1BB-2 mAb | DVVLTQSPLSLPVTLGQPASISCRSSQSLLDSDGNTYLYWFQQRPGQSP |
| | VL | RRLIYLVSNLGSGVPDRFSGSGSGTDFTLKISRVEAEDVGVYYCMQPTH |
| | | APYTFGQGTKLEIK |
| 32 | 4-1BB-2 mAb | EVQLVQSGAEVKKPGSSVKVSCKASDYTFNDYWVSWVRQAPGQGLE |
| | heavy chain | WIGEIYPNSGATNFNGKFRGRATLTVDNSASTAYMELSSLRSEDTAVY |
| | | YCTREYTRDWFAYWGQGTLVTVSSAKTTAPSVYPLAPVCGDTTGSSV |
| | | TLGCLVKGYFPEPVTLTWNSGSLSSGVHTFPAVLQSDLYTLSSSVTVTS |
| | | STWPSQSITCNVAHPASSTKVDKKIEPRGPTIKPCPPCKCPAPNLLGGPS |
| | | VFIFPPKIKDVLMISLSPIVTCVVVDVSEDDPDVQISWFVNNVEVHTAQT |
| | | QTHREDYNSTLRVVSALPIQHQDWMSGKEFKCKVNNKDLPAPIERTIS |
| | | KPKGSVRAPQVYVLPPPEEEMTKKQVTLTCMVTDFMPEDIYVEWTNN |
| | | GKTELNYKNTEPVLDSDGSYFMYSKLRVEKKNWVERNSYSCSVVHEG |
| | | LHNHHTTKSFSRTPGK |
| 33 | 4-1BB-2 mAb | DVVLTQSPLSLPVTLGQPASISCRSSQSLLDSDGNTYLYWFQQRPGQSP |
| | light chain | RRLIYLVSNLGSGVPDRFSGSGSGTDFTLKISRVEAEDVGVYYCMQPTH |
| | | APYTFGQGTKLEIKGQPKSSPSVTLFPPSSEELETNKATLVCTITDFYPGV |
| | | VTVDWKVDGTPVTQGMETTQPSKQSNNKYMASSYLTLTARAWERHS |
| | | SYSCQVTHEGHTVEKSLSRADCS |
| 34 | 4-1BB-2 × | EVQLVQSGAEVKKPGSSVKVSCKASDYTFNDYWVSWVRQAPGQGLE |
| | CCR8-1 BsAb | WIGEIYPNSGATNFNGKFRGRATLTVDNSASTAYMELSSLRSEDTAVY |
| | heavy chain | YCTREYTRDWFAYWGQGTLVTVSSAKTTAPSVYPLAPVCGDTTGSSV |
| | | TLGCLVKGYFPEPVTLTWNSGSLSSGVHTFPAVLQSDLYTLSSSVTVTS |
| | | STWPSQSITCNVAHPASSTKVDKKIEPRGPTIKPCPPCKCPAPNLLGGPS |
| | | VFIFPPKIKDVLMISLSPIVTCVVVDVSEDDPDVQISWFVNNVEVHTAQT |
| | | QTHREDYNSTLRVVSALPIQHQDWMSGKEFKCKVNNKDLPAPIERTIS |
| | | KPKGSVRAPQVYVLPPPEEEMTKKQVTLTCMVTDFMPEDIYVEWTNN |
| | | GKTELNYKNTEPVLDSDGSYFMYSKLRVEKKNWVERNSYSCSVVHEG |
| | | LHNHHTTKSFSRTPGK |
| 35 | 4-1BB-2 × | EVQLLESGGGLVQPGGSLRLSCAASGFTFSDYGVHWVRQAPGKGLEW |
| | CCR8-1 BsAb | VSGVSWNGSRTHYADSVKGRFTISRDNSKNTLYLQMNSLRAEDTAVY |
| | light chain | YCVTRGAWGQGTLVTVSSGGGGSGGGGSGGGGSGGGGSQSVLTQPPS |
| | | ASGTPGQRVTISCSGSSFNIGSHFVYWYQQLPGTAPKLLIYKNNQRPSG |
| | | VPDRFSGSKSGTSASLAISGLRSEDEADYYCAAWDDSLNGPVFGGGTK |
| | | LTVLGGGGSGGGGSGGGGS |
| | | DVVLTQSPLSLPVTLGQPASISCRSSQSLLDSDGNTYLYWFQQRPGQSP |
| | | RRLIYLVSNLGSGVPDRFSGSGSGTDFTLKISRVEAEDVGVYYCMQPTH |
| | | APYTFGQGTKLEIKADAAPTVSIFPPSSEQLTSGGASVVCFLNNFYPKDI |
| | | NVKWKIDGSERQNGVLNSWTDQDSKDSTYSMSSTLTLTKDEYERHNS |
| | | YTCEATHKTSTSPIVKSFNRNEC |
| 36 | CCR8-2 mAb | DSEMH |
| | HCDR1 | |
| 37 | CCR8-2 mAb | AIQPETGGTAYNQKFKA |
| | HCDR2 | |
| 38 | CCR8-2 mAb | RRRNFDY |
| | HCDR3 | |
| 39 | CCR8-2 mAb | RSSQSLFHSSGNTYLH |
| | LCDR1 | |
| 40 | CCR8-2 mAb | KVSNRFS |
| | LCDR2 | |
| 41 | CCR8-2 mAb | SQSTHVPFT |
| | LCDR3 | |
| 42 | CCR8-2 mAb | QVQLVQSGAEVKKPGASVKVSCKASGYTFTDSEMHWVRQATGQGLE |
| | VH | WMGAIQPETGGTAYNQKFKARVTMTRDTSISTAYMELSSLRSEDTAVY |
| | | YCARRRRNFDYWGQGTLVTVSS |
| 43 | CCR8-2 mAb | DIVMTQTPLSLSVTPGQPASISCRSSQSLFHSSGNTYLHWYLQKPGQPPQ |
| | VL | LLIYKVSNRFSGVPDRFSGSGSGTDFTLKISRVEAEDVGVYYCSQSTHV |
| | | PFTFGQGTKLEIK |
| 44 | 4-1BB-2 × | QVQLVQSGAEVKKPGASVKVSCKASGYTFTDSEMHWVRQATGQGLE |
| | CCR8-2 (LC1) | WMGAIQPETGGTAYNQKFKARVTMTRDTSISTAYMELSSLRSEDTAVYY |
| | BsAb heavy | CARRRRNFDYWGQGTLVTVSSASTKGPSVFPLAPSSKSTSGGTAALGCL |
| | chain | VKDYFPEPVTVSWNSGALTSGVHTFPAVLQSSGLYSLSSVVTVPSSSLG |
| | | TQTYICNVNHKPSNTKVDKKVEPKSCDKTHTCPPCPAPELLGGPSVFLF |
| | | PPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISK |
| | | AKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVMHEALHN |
| | | HYTQKSLSLSPGK |
| 45 | 4-1BB-2 × | DVVLTQSPLSLPVTLGQPASISCRSSQSLLDSDGNTYLYWFQQRPGQSPR |
| | CCR8-2 (LC1) | RLIYLVSNLGSGVPDRFSGSGSGTDFTLKISRVEAEDVGVYYCMQPTHA |
| | BsAb light | PYTFGQGTKLEIKGGGGSGGGGSGGGGSGGGGSEVQLVQSGAEVKKP |
| | chain | GSSVKVSCKASDYTFNDYWVSWVRQAPGQGLEWIGEIYPNSGATNFN |
| | | GKFRGRATLTVDNSASTAYMELSSLRSEDTAVYYCTREYTRDWFAYWG |
| | | QGTLVTVSSGGGGSGGGGSGGGGSDIVMTQTPLSLSVTPGQPASISCRSS |
| | | QSLFHSSGNTYLHWYLQKPGQPPQLLIYKVSNRFSGVPDRFSGSGSGTD |
| | | FTLKISRVEAEDVGVYYCSQSTHVPFTFGQGTKLEIKRTVAAPSVFIFPPS |
| | | DEQLKSGTASVVCLLNNFYPREAKVQWKVDNALQSGNSQESVTEQDS |
| | | KDSTYSLSSTLTLSKADYEKHKVYACEVTHQGLSSPVTKSFNRGEC |
| 46 | 4-1BB-2 × | QVQLVQSGAEVKKPGASVKVSCKASGYTFTDSEMHWVRQATGQGLE |
| | CCR8-2 (LC2) | WMGAIQPETGGTAYNQKFKARVTMTRDTSISTAYMELSSLRSEDTAVYY |
| | BsAb heavy | CARRRRNFDYWGQGTLVTVSSASTKGPSVFPLAPSSKSTSGGTAALGCL |
| | chain | VKDYFPEPVTVSWNSGALTSGVHTFPAVLQSSGLYSLSSVVTVPSSSLG |
| | | TQTYICNVNHKPSNTKVDKKVEPKSCDKTHTCPPCPAPELLGGPSVFLF |
| | | PPKPKDTLMISRTPEVTCVVVDVSHEDPEVKFNWYVDGVEVHNAKTK |
| | | PREEQYNSTYRVVSVLTVLHQDWLNGKEYKCKVSNKALPAPIEKTISK |
| | | AKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVMHEALHN |
| | | HYTQKSLSLSPGK |
| 47 | 4-1BB-2 × | DIVMTQTPLSLSVTPGQPASISCRSSQSLFHSSGNTYLHWYLQKPGQPPQ |
| | CCR8-2 (LC2) | LLIYKVSNRFSGVPDRFSGSGSGTDFTLKISRVEAEDVGVYYCSQSTHVP |
| | BsAb light | FTFGQGTKLEIKRTVAAPSVFIFPPSDEQLKSGTASVVCLLNNFYPREAK |
| | chain | VQWKVDNALQSGNSQESVTEQDSKDSTYSLSSTLTLSKADYEKHKVYA |
| | | CEVTHQGLSSPVTKSFNRGECGGGGSGGGGSGGGGSDVVLTQSPLSLP |
| | | VTLGQPASISCRSSQSLLDSDGNTYLYWFQQRPGQSPRRLIYLVSNLGSG |
| | | VPDRFSGSGSGTDFTLKISRVEAEDVGVYYCMQPTHAPYTFGQGTKLEI |
| | | KGGGGSGGGGSGGGGSGGGGSEVQLVQSGAEVKKPGSSVKVSCKASD |
| | | YTFNDYWVSWVRQAPGQGLEWIGEIYPNSGATNFNGKFRGRATLTVDN |
| | | SASTAYMELSSLRSEDTAVYYCTREYTRDWFAYWGQGTLVTVSS |
| 48 | 4-1BB-2 × | QVQLVQSGAEVKKPGASVKVSCKASGYTFTDSEMHWVRQATGQGLE |
| | CCR8-2 (HC) | WMGAIQPETGGTAYNQKFKARVTMTRDTSISTAYMELSSLRSEDTAVYY |
| | BsAb heavy | CARRRRNFDYWGQGTLVTVSSASTKGPSVFPLAPSSKSTSGGTAALGCL |
| | chain | VKDYFPEPVTVSWNSGALTSGVHTFPAVLQSSGLYSLSSVVTVPSSSLGT |
| | | QTYICNVNHKPSNTKVDKKVEPKSCDKTHTCPPCPAPELLGGPSVFLFP |
| | | PKPKDTLMISRTPEVTCVVVDVSHEDPEVKFNWYVDGVEVHNAKTKP |
| | | REEQYNSTYRVVSVLTVLHQDWLNGKEYKCKVSNKALPAPIEKTISKA |
| | | KGQPREPQVYTLPPSRDELTKNQVSLTCLVKGFYPSDIAVEWESNGQPE |
| | | NNYKTTPPVLDSDGSFFLYSKLTVDKSRWQQGNVFSCSVMHEALHNHY |
| | | TQKSLSLSPGKGGGGSGGGGSGGGGSDVVLTQSPLSLPVTLGQPASISC |
| | | RSSQSLLDSDGNTYLYWFQQRPGQSPRRLIYLVSNLGSGVPDRFSGSGS |
| | | GTDFTLKISRVEAEDVGVYYCMQPTHAPYTFGQGTKLEIKGGGGSGGG |
| | | GSGGGGSGGGGSEVQLVQSGAEVKKPGSSVKVSCKASDYTFNDYWVS |
| | | WVRQAPGQGLEWIGEIYPNSGATNFNGKFRGRATLTVDNSASTAYMEL |
| | | SSLRSEDTAVYYCTREYTRDWFAYWGQGTLVTVSS |
| 49 | 4-1BB-2 × | DIVMTQTPLSLSVTPGQPASISCRSSQSLFHSSGNTYLHWYLQKPGQPPQ |
| | CCR8-2 (HC) | LLIYKVSNRFSGVPDRFSGSGSGTDFTLKISRVEAEDVGVYYCSQSTHVP |
| | BsAb light | FTFGQGTKLEIKRTVAAPSVFIFPPSDEQLKSGTASVVCLLNNFYPREAK |
| | chain | VQWKVDNALQSGNSQESVTEQDSKDSTYSLSSTLTLSKADYEKHKVYA |
| | | CEVTHQGLSSPVTKSFNRGEC |
| 50 | 4-1BB-3 mAb | GYYWS |
| | HCDR1 | |
| 51 | 4-1BB-3 mAb | EINHGGYVTYNPSLES |
| | HCDR2 | |
| 52 | 4-1BB-3 mAb | DYGPGNYDWYFDL |
| | HCDR3 | |
| 53 | 4-1BB-3 mAb | RASQSVSSYLA |
| | LCDR1 | |
| 54 | 4-1BB-3 mAb | DASNRAT |
| | LCDR2 | |
| 55 | 4-1BB-3 mAb | QQRSNWPPALT |
| | LCDR3 | |
| 56 | 4-1BB-3 mAb | QVQLQQWGAGLLKPSETLSLTCAVYGGSFSGYYWSWIRQSPEKGLEWI |
| | VH | GEINHGGYVTYNPSLESRVTISVDTSKNQFSLKLSSVTAADTAVYYCAR |
| | | DYGPGNYDWYFDLWGRGTLVTVSS |
| 57 | 4-1BB-3 mAb | EIVLTQSPATLSLSPGERATLSCRASQSVSSYLAWYQQKPGQAPRLLIYD |
| | VL | ASNRATGIPARFSGSGSGTDFTLTISSLEPEDFAVYYCQQRSNWPPALTF |
| | | CGGTKVEIK |
| 58 | CCR8-3 mAb | TYAMN |
| | HCDR1 | |
| 59 | CCR8-3 mAb | RIRSKANNYATYYADSVKD |
| | HCDR2 | |
| 60 | CCR8-3 mAb | DRSRGEDYAMDY |
| | HCDR3 | |
| 61 | CCR8-3 mAb | RSSKSLLHSNANTYLY |
| | LCDR1 | |
| 62 | CCR8-3 mAb | RMSNLAS |
| | LCDR2 | |
| 63 | CCR8-3 mAb | MQHLEYPFT |
| | LCDR3 | |
| 64 | CCR8-3 mAb | EVQLVESGGGLVQPGGSLKLSCAASGFTFNTYAMNWVRQASGKGLEW |
| | VH | VARIRSKANNYATYYADSVKDRFTISRDDSKNTLYLQMNNLKTEDTAV |
| | | YYCVRDRSRGEDYAMDYWGQGTLVTVSS |
| 65 | CCR8-3 mAb | DIVMTQSPLSLPVTPGEPASISCRSSKSLLHSNANTYLYWFLQKPGQSPQ |
| | VL | LLIYRMSNLASGVPDRESGSGSGTAFTLKISRVEAEDVGVYYCMQHLE |
| | | YPFTFGQGTKLEIK |
| 66 | 4-1BB-3 × | QVQLQQWGAGLLKPSETLSLTCAVYGGSFSGYYWSWIRQSPEKGLEWI |
| | CCR8-3 BsAb | GEINHGGYVTYNPSLESRVTISVDTSKNQFSLKLSSVTAADTAVYYCAR |
| | heavy chain | DYGPGNYDWYFDLWGRGTLVTVSSASTKGPSVFPLAPSSKSTSGGTAA |
| | | LGCLVKDYFPEPVTVSWNSGALTSGVHTFPAVLQSSGLYSLSSVVTVPS |
| | | SSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKT |
| | | ISKAKGQPREPQVYTLPPSRDELTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPGK |
| 67 | 4-1BB-3 × | DIVMTQSPLSLPVTPGEPASISCRSSKSLLHSNANTYLYWFLQKPGQSPQ |
| | CCR8-3 BsAb | LLIYRMSNLASGVPDRFSGSGSGTAFTLKISRVEAEDVGVYYCMQHLEY |
| | light chain | PFTFGQGTKLEIKGGGGSGGGGSGGGGSGGGGSEVQLVESGGGLVQPG |
| | | GSLKLSCAASGFTFNTYAMNWVRQASGKGLEWVARIRSKANNYATYY |
| | | ADSVKDRFTISRDDSKNTLYLQMNNLKTEDTAVYYCVRDRSRGEDYAM |
| | | DYWGQGTLVTVSSGGGGSGGGGSGGGGSEIVLTQSPATLSLSPGERATL |
| | | SCRASQSVSSYLAWYQQKPGQAPRLLIYDASNRATGIPARFSGSGSGTD |
| | | FTLTISSLEPEDFAVYYCQQRSNWPPALTFCGGTKVEIKRTVAAPSVFIFPP |
| | | SDEQLKSGTASVVCLLNNFYPREAKVQWKVDNALQSGNSQESVTEQD |
| | | SKDSTYSLSSTLTLSKADYEKHKVYACEVTHQGLSSPVTKSFNRGEC |
| 68 | 4-1BB-4 mAb | TYWIS |
| | HCDR1 | |
| 69 | 4-1BB-4 mAb | KIYPGDSYTNYSPSFQG |
| | HCDR2 | |
| 70 | 4-1BB-4 mAb | GYGIFDY |
| | HCDR3 | |
| 71 | 4-1BB-4 mAb | SGDNIGDQYAH |
| | LCDR1 | |
| 72 | 4-1BB-4 mAb | QDKNRPS |
| | LCDR2 | |
| 73 | 4-1BB-4 mAb | ATYTGFGSLAV |
| | LCDR3 | |
| 74 | 4-1BB-4 mAb | EVQLVQSGAEVKKPGESLRISCKGSGYSFSTYWISWVRQMPGKGLEW |
| | VH | MGKIYPGDSYTNYSPSFQGQVTISADKSISTAYLQWSSLKASDTAMYY |
| | | CARGYGIFDYWGQGTLVTVSS |
| 75 | 4-1BB-4 mAb | SYELTQPPSVSVSPGQTASITCSGDNIGDQYAHWYQQKPGQSPVLVIYQ |
| | VL | DKNRPSGIPERFSGSNSGNTATLTISGTQAMDEADYYCATYTGFGSLAV |
| | | FGGGTKLTVL |
| 76 | CCR8-4 mAb | NAVMY |
| | HCDR1 | |
| 77 | CCR8-4 mAb | RIKTKENNYATYYADAVKG |
| | HCDR2 | |
| 78 | CCR8-4 mAb | GDRNKPFAY |
| | HCDR3 | |
| 79 | CCR8-4 mAb | RASTSVITLLH |
| | LCDR1 | |
| 80 | CCR8-4 mAb | GASNLES |
| | LCDR2 | |
| 81 | CCR8-4 mAb | QQSWNDPYT |
| | LCDR3 | |
| 82 | CCR8-4 mAb | EVQLVESGGGLVQPGGSLKLSCAASGFIFSNAVMYWVRQASGKGLEW |
| | VH | VARIKTKENNYATYYADAVKGRFTISRDDSKNMVYLQMNSLKTEDTA |
| | | VYYCTAGDRNKPFAYWGQGTLVTVSS |
| 83 | CCR8-4 mAb | ETVVTQSPATLSLSPGERATLSCRASTSVITLLHWFQQKPGQAPRLLIHG |
| | VL | ASNLESRVPARFSGSGSGTDFTLTISSLEPEDFATYFCQQSWNDPYTFGQ |
| | | GTKLEIK |
| 84 | 4-1BB-4 × | EVQLVESGGGLVQPGGSLKLSCAASGFIFSNAVMYWVRQASGKGLEW |
| | CCR8-3 BsAb | VARIKTKENNYATYYADAVKGRFTISRDDSKNMVYLQMNSLKTEDTA |
| | heavy chain | VYYCTAGDRNKPFAYWGQGTLVTVSSASTKGPSVFPLAPSSKSTSGGT |
| | | AALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAP |
| | | IEKTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVMH |
| | | EALHNHYTQKSLSLSPGK |
| 85 | 4-1BB-4 × | SYELTQPPSVSVSPGQTASITCSGDNIGDQYAHWYQQKPGQSPVLVIYQ |
| | CCR8-3 BsAb | DKNRPSGIPERFSGSNSGNTATLTISGTQAMDEADYYCATYTGFGSLAVF |
| | light chain | GGGTKLTVLGGGGSGGGGSGGGGSGGGGSEVQLVQSGAEVKKPGESL |
| | | RISCKGSGYSFSTYWISWVRQMPGKGLEWMGKIYPGDSYTNYSPSFQG |
| | | QVTISADKSISTAYLQWSSLKASDTAMYYCARGYGIFDYWGQGTLVTV |
| | | SSGGGGSGGGGSGGGGSETVVTQSPATLSLSPGERATLSCRASTSVITLL |
| | | HWFQQKPGQAPRLLIHGASNLESRVPARFSGSGSGTDFTLTISSLEPEDF |
| | | ATYFCQQSWNDPYTFGQGTKLEIKRTVAAPSVFIFPPSDEQLKSGTASVV |
| | | CLLNNFYPREAKVQWKVDNALQSGNSQESVTEQDSKDSTYSLSSTLTL |
| | | SKADYEKHKVYACEVTHQGLSSPVTKSFNRGEC |
| 86 | Linker 2 | GGGGSGGGGSGGGGS |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An antibody or antigen-binding fragment thereof, comprising:
i) a first antigen-binding domain that specifically binds to 4-1BB; and
ii) a second antigen-binding domain that specifically binds to C—C motif chemokine receptor 8 (CCR8).

2. The antibody or antigen-binding fragment thereof of claim 1, wherein the 4-1BB is human 4-1BB, and wherein the CCR8 is human CCR8.

3. The antibody or antigen-binding fragment thereof of claim 1, wherein the first antigen-binding domain comprises a first heavy chain variable region (VH1) and a first light chain variable region (VL1); and the second antigen-binding domain comprises a second heavy chain variable region (VH2) and a second light chain variable region (VL2).

4. The antibody or antigen-binding fragment thereof of claim 1, wherein the second antigen-binding domain is a single-chain fragment variable (scFv) domain, wherein the VH2 and VL2 are linked by a first linker.

5. The antibody or antigen-binding fragment thereof of claim 4, wherein the second antigen-binding domain is connected to the N-terminus of a light chain through a second linker.

6. The antibody or antigen-binding fragment thereof of claim 1, wherein the antibody or antigen-binding fragment thereof is a bispecific antibody or antigen-binding fragment thereof.

7. The antibody or antigen-binding fragment thereof of claim 1, further comprising an Fc region.

8. The antibody or antigen-binding fragment thereof of claim 7, wherein the C-terminus of the VH1 of the first antigen-binding domain is connected to the Fc region, optionally through a CH1 domain.

9. The antibody or antigen-binding fragment thereof of claim 3, wherein the antibody comprises a first heavy chain comprising the VH1 and a first light chain comprising the VL1; and a second heavy chain comprising the VH2 and a second light chain comprising the VL2.

10. The antibody or antigen-binding fragment thereof of claim 7, wherein the Fc region is an Fc region of human IgG1, IgG2, IgG3, or IgG4.

11. The antibody or antigen-binding fragment thereof of claim 10, wherein the Fc region is an Fc region of human IgG1 or IgG2.

12. The antibody or antigen-binding fragment thereof of claim 11, wherein the Fc region is a wild type Fc, or an Fc with enhanced ADCC activity, or an Fc with enhanced ADCP activity.

13. The antibody or antigen-binding fragment thereof of claim 3, wherein the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, 3 amino acid sequences and the selected VL1 CDRs 1, 2, 3 amino acid sequences are one of the following:
(1) the selected VH1 CDRs 1, 2, 3 amino acid sequences are set forth in SEQ ID NOs: 1, 2 and 3, respectively, and the selected VL1 CDRs 1, 2, 3 amino acid sequences are set forth in SEQ ID NOs: 4, 5 and 6, respectively;
(2) the selected VH1 CDRs 1, 2, 3 amino acid sequences are set forth in SEQ ID NOs: 24, 25 and 26, respectively, and the selected VL1 CDRs 1, 2, 3 amino acid sequences are set forth in SEQ ID NOs: 27, 28 and 29, respectively;
(3) the selected VH1 CDRs 1, 2, 3 amino acid sequences are set forth in SEQ ID NOs: 50, 51 and 52, respectively, and the selected VL1 CDRs 1, 2, 3 amino acid sequences are set forth in SEQ ID NOs: 53, 54 and 55, respectively; and
(4) the selected VH1 CDRs 1, 2, 3 amino acid sequences are set forth in SEQ ID NOs: 68, 69 and 70, respectively, and the selected VL1 CDRs 1, 2, 3 amino acid sequences are set forth in SEQ ID NOs: 71, 72 and 73, respectively.

14. The antibody or antigen-binding fragment thereof of claim 3, wherein the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and
the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence, wherein the selected VH2 CDRs 1, 2, 3 amino acid sequences and the selected VL2 CDRs 1, 2, 3 amino acid sequences are one of the following:
(1) the selected VH2 CDRs 1, 2, 3 amino acid sequences are set forth in SEQ ID NOs: 11, 12 and 13, respectively, and the selected VL2 CDRs 1, 2, 3 amino acid sequences are set forth in SEQ ID NOs: 14, 15 and 16, respectively;
(2) the selected VH2 CDRs 1, 2, 3 amino acid sequences are set forth in SEQ ID NOs: 36, 37 and 38, respectively, and the selected VL2 CDRs 1, 2, 3 amino acid sequences are set forth in SEQ ID NOs: 39, 40 and 41, respectively
(3) the selected VH2 CDRs 1, 2, 3 amino acid sequences are set forth in SEQ ID NOs: 58, 59 and 60, respectively, and the selected VL2 CDRs 1, 2, 3 amino acid sequences are set forth in SEQ ID NOs: 61, 62 and 63, respectively; and
(4) the selected VH2 CDRs 1, 2, 3 amino acid sequences are set forth in SEQ ID NOs: 76, 77 and 78, respectively, and the selected VL2 CDRs 1, 2, 3 amino acid sequences are set forth in SEQ ID NOs: 79, 80 and 81, respectively.

15. The antibody or antigen-binding fragment thereof of claim 3, wherein
the selected VH1 CDRs 1, 2, 3 amino acid sequences are set forth in SEQ ID NOs: 1, 2 and 3, respectively, the selected VL1 CDRs 1, 2, 3 amino acid sequences are set forth in SEQ ID NOs: 4, 5 and 6, respectively; the selected VH2 CDRs 1, 2, 3 amino acid sequences are set forth in SEQ ID NOs: 11 12 and 13, respectively, and the selected VL2 CDRs 1, 2, 3 amino acid sequences are set forth in SEQ ID NOs: 14, 15 and 16, respectively.

16. An antibody or antigen-binding fragment thereof that cross-competes with the antibody or antigen-binding fragment thereof of claim 1.

17. A method of treating a subject having cancer, the method comprising administering a therapeutically effective amount of a composition comprising the antibody or antigen-binding fragment thereof of claim 1 to the subject.

18. The method of claim 17, wherein the cancer is a solid tumor or a blood tumor.

19. The method of claim 18, wherein the cancer is breast cancer, uterine corpus cancer, cervical cancer, ovary cancer, prostate cancer, lung cancer, stomach cancer, non-small cell lung cancer, spleen cancer, head and neck squamous cell carcinoma, esophageal cancer, bladder cancer, melanoma, colorectal cancer, kidney cancer, non-Hodgkin lymphoma, urothelial cancer, sarcoma, blood cell cancer such as leukemia or lymphoma, bile duct carcinoma, gallbladder carcinoma, thyroid carcinoma, testicular carcinoma, thymic carcinoma, or hepatocarcinoma.

20. The method of claim 19, wherein the cancer is breast cancer, uterine corpus cancer, ovary cancer, lung cancer, colorectal cancer, kidney cancer and sarcoma.

21. The method of claim 18, wherein the cancer is colon cancer.

22. The method of claim 17, wherein the subject is further treated with an effective amount of an anti-OX40 antibody, an anti-PD-1 antibody, an anti-CTLA4 antibody, an anti-CD40 antibody, or an anti-PD-L1 antibody.

23. A method of decreasing the rate of tumor growth, the method comprising
contacting a tumor cell with an effective amount of a composition comprising an antibody or antigen-binding fragment thereof of claim 1.

24. A method of killing a tumor cell, the method comprising
Contacting a tumor cell with an effective amount of a composition comprising the antibody or antigen-binding fragment thereof of claim 1.

25. A method of increasing immune response in a subject, the method comprising
Administering to the subject an effective amount of a composition comprising the antibody or antigen-binding fragment thereof of claim 1.

26. A pharmaceutical composition comprising the antibody or antigen-binding fragment thereof of claim 1, and a pharmaceutically acceptable carrier.


SEQUENCE LISTING (86 sequences)

SEQ ID NO: 1 - protein, 5 aa, synthetic construct
SDGVH

SEQ ID NO: 2 - protein, 16 aa, synthetic construct
IIYYDGGTDYNSAIKS

SEQ ID NO: 3 - protein, 5 aa, synthetic construct
IHFDY

SEQ ID NO: 4 - protein, 10 aa, synthetic construct
RASSSVSYMY

SEQ ID NO: 5 - protein, 7 aa, synthetic construct
DTSKLAS

SEQ ID NO: 6 - protein, 9 aa, synthetic construct
QQWSSTPLT

SEQ ID NO: 7 - protein, 113 aa, synthetic construct
QVQLKEAGPGLVQPTQTLSLTCTVSGFSLTSDGVHWIRQPPGKGLEWMGIIYYDGGTDYNSAIKSRLSISRDTSKSQVFLKINSLQTDDTAMYYCARIHFDYWGQGVMVTVSS

SEQ ID NO: 8 - protein, 107 aa, synthetic construct
DIVLTQSPTTIAASPGEKVTITCRASSSVSYMYWYQQKSGASPKLWIYDTSKLASGVPNRFSGSGSGTSYSLAINTMETEDAATYYCQQWSSTPLTFGSGTKLEIKR

SEQ ID NO: 9 - protein, 443 aa, synthetic construct
QVQLKEAGPGLVQPTQTLSLTCTVSGFSLTSDGVHWIRQPPGKGLEWMGIIYYDGGTDYNSAIKSRLSISRDTSKSQVFLKINSLQTDDTAMYYCARIHFDYWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 10 - protein, 213 aa, synthetic construct
DIVLTQSPTTIAASPGEKVTITCRASSSVSYMYWYQQKSGASPKLWIYDTSKLASGVPNRFSGSGSGTSYSLAINTMETEDAATYYCQQWSSTPLTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 11 - protein, 5 aa, synthetic construct
DYGVH

SEQ ID NO: 12 - protein, 17 aa, synthetic construct
GVSWNGSRTHYADSVKG

SEQ ID NO: 13 - skipped

SEQ ID NO: 14 - protein, 13 aa, synthetic construct
SGSSFNIGSHFVY

SEQ ID NO: 15 - protein, 7 aa, synthetic construct
KNNQRPS

SEQ ID NO: 16 - protein, 11 aa, synthetic construct
AAWDDSLNGPV

SEQ ID NO: 17 - protein, 112 aa, synthetic construct
EVQLLESGGGLVQPGGSLRLSCAASGFTFSDYGVHWVRQAPGKGLEWVSGVSWNGSRTHYADSVKGRFTISRDNSKNTLYLQMNSLRAEDTAVYYCVTRGAWGQGTLVTVSS

SEQ ID NO: 18 - protein, 110 aa, synthetic construct
QSVLTQPPSASGTPGQRVTISCSGSSFNIGSHFVYWYQQLPGTAPKLLIYKNNQRPSGVPDRFSGSKSGTSASLAISGLRSEDEADYYCAAWDDSLNGPVFGGGTKLTVL

SEQ ID NO: 19 - protein, 442 aa, synthetic construct
EVQLLESGGGLVQPGGSLRLSCAASGFTFSDYGVHWVRQAPGKGLEWVSGVSWNGSRTHYADSVKGRFTISRDNSKNTLYLQMNSLRAEDTAVYYCVTRGAWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 20 - protein, 216 aa, synthetic construct
QSVLTQPPSASGTPGQRVTISCSGSSFNIGSHFVYWYQQLPGTAPKLLIYKNNQRPSGVPDRFSGSKSGTSASLAISGLRSEDEADYYCAAWDDSLNGPVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 21 - protein, 443 aa, synthetic construct; identical to SEQ ID NO: 9

SEQ ID NO: 22 - protein, 470 aa, synthetic construct
EVQLLESGGGLVQPGGSLRLSCAASGFTFSDYGVHWVRQAPGKGLEWVSGVSWNGSRTHYADSVKGRFTISRDNSKNTLYLQMNSLRAEDTAVYYCVTRGAWGQGTLVTVSSGGGGSGGGGSGGGGSGGGGSQSVLTQPPSASGTPGQRVTISCSGSSFNIGSHFVYWYQQLPGTAPKLLIYKNNQRPSGVPDRFSGSKSGTSASLAISGLRSEDEADYYCAAWDDSLNGPVFGGGTKLTVLGGGGSGGGGSGGGGSDIVLTQSPTTIAASPGEKVTITCRASSSVSYMYWYQQKSGASPKLWIYDTSKLASGVPNRFSGSGSGTSYSLAINTMETEDAATYYCQQWSSTPLTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 23 - protein, 20 aa, synthetic construct
GGGGSGGGGSGGGGSGGGGS

SEQ ID NO: 24 - protein, 5 aa, synthetic construct
DYWVS

SEQ ID NO: 25 - protein, 17 aa, synthetic construct
EIYPNSGATNFNGKFRG

SEQ ID NO: 26 - protein, 9 aa, synthetic construct
EYTRDWFAY

SEQ ID NO: 27 - protein, 16 aa, synthetic construct
RSSQSLLDSDGNTYLY

SEQ ID NO: 28 - protein, 7 aa, synthetic construct
LVSNLGS

SEQ ID NO: 29 - protein, 9 aa, synthetic construct
MQPTHAPYT

SEQ ID NO: 30 - protein, 118 aa, synthetic construct
EVQLVQSGAEVKKPGSSVKVSCKASDYTFNDYWVSWVRQAPGQGLEWIGEIYPNSGATNFNGKFRGRATLTVDNSASTAYMELSSLRSEDTAVYYCTREYTRDWFAYWGQGTLVTVSS

SEQ ID NO: 31 - protein, 112 aa, synthetic construct
DVVLTQSPLSLPVTLGQPASISCRSSQSLLDSDGNTYLYWFQQRPGQSPRRLIYLVSNLGSGVPDRFSGSGSGTDFTLKISRVEAEDVGVYYCMQPTHAPYTFGQGTKLEIK

SEQ ID NO: 32 - protein, 448 aa, synthetic construct
EVQLVQSGAEVKKPGSSVKVSCKASDYTFNDYWVSWVRQAPGQGLEWIGEIYPNSGATNFNGKFRGRATLTVDNSASTAYMELSSLRSEDTAVYYCTREYTRDWFAYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 33 - protein, 218 aa, synthetic construct
DVVLTQSPLSLPVTLGQPASISCRSSQSLLDSDGNTYLYWFQQRPGQSPRRLIYLVSNLGSGVPDRFSGSGSGTDFTLKISRVEAEDVGVYYCMQPTHAPYTFGQGTKLEIKGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 34 - protein, 448 aa, synthetic construct; identical to SEQ ID NO: 32

SEQ ID NO: 35 - protein, 475 aa, synthetic construct
EVQLLESGGGLVQPGGSLRLSCAASGFTFSDYGVHWVRQAPGKGLEWVSGVSWNGSRTHYADSVKGRFTISRDNSKNTLYLQMNSLRAEDTAVYYCVTRGAWGQGTLVTVSSGGGGSGGGGSGGGGSGGGGSQSVLTQPPSASGTPGQRVTISCSGSSFNIGSHFVYWYQQLPGTAPKLLIYKNNQRPSGVPDRFSGSKSGTSASLAISGLRSEDEADYYCAAWDDSLNGPVFGGGTKLTVLGGGGSGGGGSGGGGSDVVLTQSPLSLPVTLGQPASISCRSSQSLLDSDGNTYLYWFQQRPGQSPRRLIYLVSNLGSGVPDRFSGSGSGTDFTLKISRVEAEDVGVYYCMQPTHAPYTFGQGTKLEIKADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 36 - protein, 5 aa, synthetic construct
DSEMH

SEQ ID NO: 37 - protein, 17 aa, synthetic construct
AIQPETGGTAYNQKFKA

SEQ ID NO: 38 - protein, 7 aa, synthetic construct
RRRNFDY

SEQ ID NO: 39 - protein, 16 aa, synthetic construct
RSSQSLFHSSGNTYLH

SEQ ID NO: 40 - protein, 7 aa, synthetic construct
KVSNRFS

SEQ ID NO: 41 - protein, 9 aa, synthetic construct
SQSTHVPFT

SEQ ID NO: 42 - protein, 116 aa, synthetic construct
QVQLVQSGAEVKKPGASVKVSCKASGYTFTDSEMHWVRQATGQGLEWMGAIQPETGGTAYNQKFKARVTMTRDTSISTAYMELSSLRSEDTAVYYCARRRRNFDYWGQGTLVTVSS

SEQ ID NO: 43 - protein, 112 aa, synthetic construct
DIVMTQTPLSLSVTPGQPASISCRSSQSLFHSSGNTYLHWYLQKPGQPPQLLIYKVSNRFSGVPDRFSGSGSGTDFTLKISRVEAEDVGVYYCSQSTHVPFTFGQGTKLEIK

SEQ ID NO: 44 - protein, 446 aa, synthetic construct
QVQLVQSGAEVKKPGASVKVSCKASGYTFTDSEMHWVRQATGQGLEWMGAIQPETGGTAYNQKFKARVTMTRDTSISTAYMELSSLRSEDTAVYYCARRRRNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 484 aa, synthetic construct
DVVLTQSPLSLPVTLGQPASISCRSSQSLLDSDGNTYLYWFQQRPGQSPRRLIYLVSNLGSGVPDRFSGSGSGTDFTLKISRVEAEDVGVYYCMQPTHAPYTFGQGTKLEIKGGGGSGGGGSGGGGSGGGGSEVQLVQSGAEVKKPGSSVKVSCKASDYTFNDYWVSWVRQAPGQGLEWIGEIYPNSGATNFNGKFRGRATLTVDNSASTAYMELSSLRSEDTAVYYCTREYTRDWFAYWGQGTLVTVSSGGGGSGGGGSGGGGSDIVMTQTPLSLSVTPGQPASISCRSSQSLFHSSGNTYLHWYLQKPGQPPQLLIYKVSNRFSGVPDRFSGSGSGTDFTLKISRVEAEDVGVYYCSQSTHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 446 aa, synthetic construct; identical to SEQ ID NO: 44

SEQ ID NO: 47 - protein, 484 aa, synthetic construct
DIVMTQTPLSLSVTPGQPASISCRSSQSLFHSSGNTYLHWYLQKPGQPPQLLIYKVSNRFSGVPDRFSGSGSGTDFTLKISRVEAEDVGVYYCSQSTHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSDVVLTQSPLSLPVTLGQPASISCRSSQSLLDSDGNTYLYWFQQRPGQSPRRLIYLVSNLGSGVPDRFSGSGSGTDFTLKISRVEAEDVGVYYCMQPTHAPYTFGQGTKLEIKGGGGSGGGGSGGGGSGGGGSEVQLVQSGAEVKKPGSSVKVSCKASDYTFNDYWVSWVRQAPGQGLEWIGEIYPNSGATNFNGKFRGRATLTVDNSASTAYMELSSLRSEDTAVYYCTREYTRDWFAYWGQGTLVTVSS

SEQ ID NO: 48 - protein, 711 aa, synthetic construct
QVQLVQSGAEVKKPGASVKVSCKASGYTFTDSEMHWVRQATGQGLEWMGAIQPETGGTAYNQKFKARVTMTRDTSISTAYMELSSLRSEDTAVYYCARRRRNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDVVLTQSPLSLPVTLGQPASISCRSSQSLLDSDGNTYLYWFQQRPGQSPRRLIYLVSNLGSGVPDRFSGSGSGTDFTLKISRVEAEDVGVYYCMQPTHAPYTFGQGTKLEIKGGGGSGGGGSGGGGSGGGGSEVQLVQSGAEVKKPGSSVKVSCKASDYTFNDYWVSWVRQAPGQGLEWIGEIYPNSGATNFNGKFRGRATLTVDNSASTAYMELSSLRSEDTAVYYCTREYTRDWFAYWGQGTLVTVSS

SEQ ID NO: 49 - protein, 219 aa, synthetic construct
DIVMTQTPLSLSVTPGQPASISCRSSQSLFHSSGNTYLHWYLQKPGQPPQLLIYKVSNRFSGVPDRFSGSGSGTDFTLKISRVEAEDVGVYYCSQSTHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 5 aa, synthetic construct
GYYWS

SEQ ID NO: 51 - protein, 16 aa, synthetic construct
EINHGGYVTYNPSLES

SEQ ID NO: 52 - protein, 13 aa, synthetic construct
DYGPGNYDWYFDL

SEQ ID NO: 53 - protein, 11 aa, synthetic construct
RASQSVSSYLA

SEQ ID NO: 54 - protein, 7 aa, synthetic construct
DASNRAT

SEQ ID NO: 55 - protein, 11 aa, synthetic construct
QQRSNWPPALT

SEQ ID NO: 56 - protein, 121 aa, synthetic construct [sequence omitted]

SEQ ID NO: 57 - protein, 109 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFCGGTKVEIK

SEQ ID NO: 58 - protein, 5 aa, synthetic construct
TYAMN

SEQ ID NO: 59 - protein, 19 aa, synthetic construct
RIRSKANNYATYYADSVKD

SEQ ID NO: 60 - protein, 12 aa, synthetic construct
DRSRGEDYAMDY

SEQ ID NO: 61 - protein, 16 aa, synthetic construct
RSSKSLLHSNANTYLY

SEQ ID NO: 62 - protein, 7 aa, synthetic construct
RMSNLAS

SEQ ID NO: 63 - protein, 9 aa, synthetic construct
MQHLEYPFT

SEQ ID NO: 64 - protein, 123 aa, synthetic construct
EVQLVESGGGLVQPGGSLKLSCAASGFTFNTYAMNWVRQASGKGLEWVARIRSKANNYATYYADSVKDRFTISRDDSKNTLYLQMNNLKTEDTAVYYCVRDRSRGEDYAMDYWGQGTLVTVSS

SEQ ID NO: 65 - protein, 112 aa, synthetic construct
DIVMTQSPLSLPVTPGEPASISCRSSKSLLHSNANTYLYWFLQKPGQSPQLLIYRMSNLASGVPDRFSGSGSGTAFTLKISRVEAEDVGVYYCMQHLEYPFTFGQGTKLEIK

SEQ ID NO: 66 - protein, 446 aa, synthetic construct; identical to SEQ ID NO: 44

SEQ ID NO: 67 - protein, 484 aa, synthetic construct; identical to SEQ ID NO: 45

SEQ ID NO: 68 - protein, 5 aa, synthetic construct
TYWIS

SEQ ID NO: 69 - protein, 17 aa, synthetic construct
KIYPGDSYTNYSPSFQG

SEQ ID NO: 70 - protein, 7 aa, synthetic construct
GYGIFDY

SEQ ID NO: 71 - protein, 11 aa, synthetic construct
SGDNIGDQYAH

SEQ ID NO: 72 - protein, 7 aa, synthetic construct
QDKNRPS

SEQ ID NO: 73 - protein, 11 aa, synthetic construct
ATYTGFGSLAV

SEQ ID NO: 74 - protein, 116 aa, synthetic construct
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSS

SEQ ID NO: 75 - protein, 108 aa, synthetic construct
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVL

SEQ ID NO: 76 - protein, 5 aa, synthetic construct
NAVMY

SEQ ID NO: 77 - protein, 19 aa, synthetic construct
RIKTKFNNYATYYADAVKG

SEQ ID NO: 78 - protein, 9 aa, synthetic construct
GDRNKPFAY

SEQ ID NO: 79 - protein, 11 aa, synthetic construct
RASTSVITLLH

SEQ ID NO: 80 - protein, 7 aa, synthetic construct
GASNLES

SEQ ID NO: 81 - protein, 9 aa, synthetic construct
QQSWNDPYT

SEQ ID NO: 82 - protein, 120 aa, synthetic construct
EVQLVESGGGLVQPGGSLKLSCAASGFIFSNAVMYWVRQASGKGLEWVARIKTKFNNYATYYADAVKGRFTISRDDSKNMVYLQMNSLKTEDTAVYYCTAGDRNKPFAYWGQGTLVTVSS

SEQ ID NO: 83 - protein, 107 aa, synthetic construct
ETVVTQSPATLSLSPGERATLSCRASTSVITLLHWFQQKPGQAPRLLIHGASNLESRVPARFSGSGSGTDFTLTISSLEPEDFATYFCQQSWNDPYTFGQGTKLEIK

SEQ ID NO: 84 - protein, 446 aa, synthetic construct; identical to SEQ ID NO: 44

SEQ ID NO: 85 - protein, 484 aa, synthetic construct; identical to SEQ ID NO: 45

SEQ ID NO: 86 - protein, 15 aa, synthetic construct
GGGGSGGGGSGGGGS